(19) United States
(12) Patent Application Publication
(10) Pub. No.: US 2025/0099483 A1
(43) Pub. Date: Mar. 27, 2025

(54) THERAPEUTIC COMPOUNDS

(71) Applicant: ARMGO Pharma, Inc., Ardsley, NY (US)

(72) Inventor: Eugene E. MARCANTONIO, Tenafly, NJ (US)

(21) Appl. No.: 18/662,117

(22) Filed: May 13, 2024

Related U.S. Application Data
(63) Continuation of application No. PCT/US22/50163, filed on Nov. 16, 2022.
(60) Provisional application No. 63/280,067, filed on Nov. 16, 2021.

(51) Int. Cl. A61K 31/554; A61K 31/4458; A61K 45/06

(52) U.S. Cl. CPC A61K 31/554; A61K 31/4458; A61K 45/06

(57) ABSTRACT

The present disclosure provides methods of treating catecholaminergic polymorphic ventricular tachycardia, comprising administering a pharmaceutical composition comprising, in a unit dosage form, a therapeutically effective amount of 4-[(7-methoxy-2,3-dihydro-1,4-benzothiazepin-1(5H)yl)methyl]benzoic acid hemifumarate, and a pharmaceutically acceptable excipient.

### CROSS REFERENCE

This application is a continuation of International Patent Application No. PCT/US2022/050163, filed Nov. 16, 2022, which claims the benefit of U.S. Provisional Application No. 63/280,067, filed Nov. 16, 2021, each of which is incorporated by reference herein in its entirety.

### STATEMENT REGARDING GOVERNMENT SUPPORT

This invention was made with government support under contract number R01FD007279 awarded by the Food and Drug Administration. The government has certain rights in the invention.

### BACKGROUND

The sarcoplasmic reticulum (SR) is a structure in cells that functions, among other things, as a specialized intracellular calcium (Ca²⁺) store. Ryanodine receptors (RyRs) are channels in the SR that open and close to regulate the release of Ca²⁺ from the SR into the intracellular cytoplasm of the cell. Release of Ca²⁺ into the cytoplasm from the SR increases cytoplasmic Ca²⁺ concentration. Open probability of RyRs refers to the likelihood that a RyR is open at any given moment, and therefore capable of releasing Ca²⁺ into the cytoplasm from the SR. Three RyR isoforms are known. RyR1 is the predominant isoform expressed in mammalian skeletal muscle, RyR2 is predominantly found in cardiac muscle, whereas RyR3 expression is low in skeletal muscle.

Ca²⁺ release from the SR is modulated by several RyR binding proteins. Calstabin1 (FKBP12) and Calstabin2 (FKBP12.6) stabilize the closed state of the RyR1 and RyR2, respectively. Mutations in RYR1 or RYR2 are characterized by reduced binding of Calstabin1 or Calstabin2, respectively, and inappropriate channel opening not related to contraction signals. This channel opening is further exacerbated by post-translational modifications such as PKA-phosphorylation, oxidation, or nitrosylation of the RyR channel. The resulting dissociation of Calstabin can lead to leaky channels, which exhibit a pathologic increase in the open probability under resting conditions. The SR Ca²⁺ leak leads to a reduction in SR Ca²⁺ content, with less Ca²⁺ available for release and consequently weaker muscle contractions.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, the present disclosure provides a method of treating catecholaminergic polymorphic ventricular tachycardia (CPVT), comprising administering to a subject in need thereof a therapeutically-effective amount of a compound that is 4-[(7-methoxy-2,3-dihydro-1,4-benzothiazepin-4(5H)yl)methyl]benzoic acid or a pharmaceutically-acceptable salt thereof, wherein the administering is once daily.

### DETAILED DESCRIPTION OF THE DRAWINGS

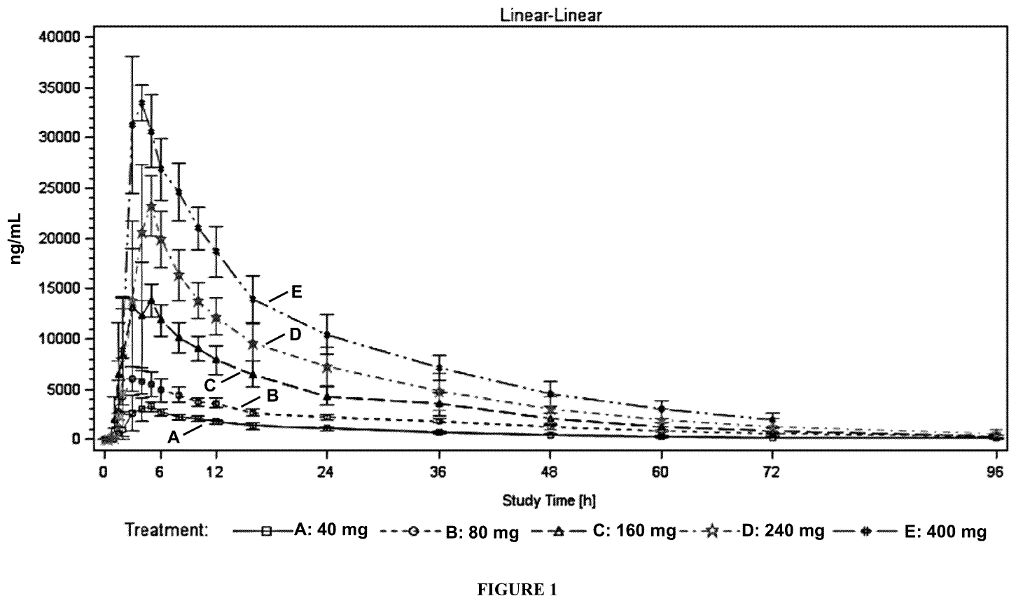

FIG. 1 is a chart depicting the time profile of Compound 1 mean plasma concentration (+/−SD) on Day 1 of Part I of the study detailed in EXAMPLE 6.

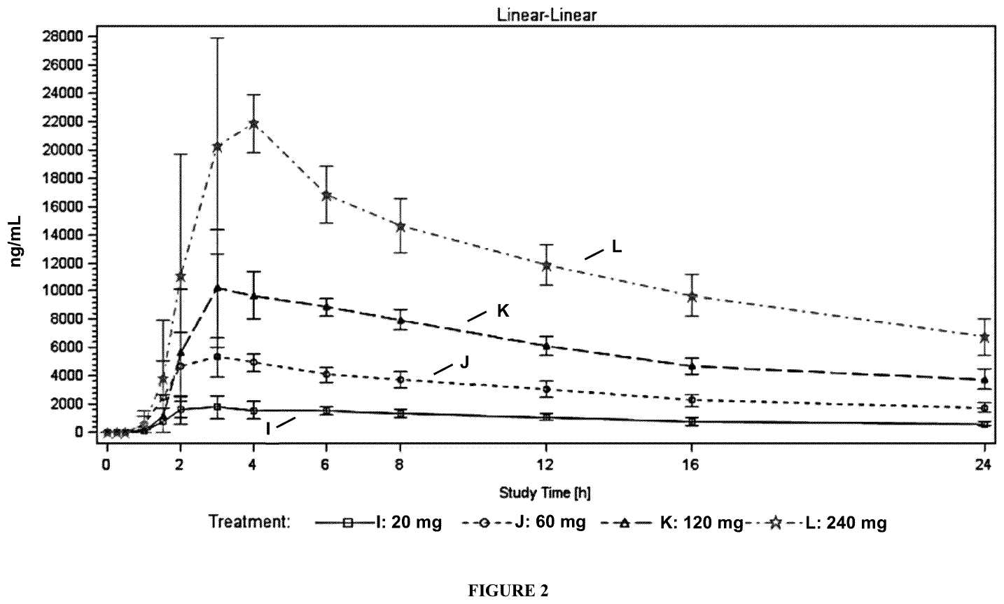

FIG. 2 is a chart depicting the time profile of Compound 1 mean plasma concentration (+/−SD) on Day 1 of Part II of the study detailed in EXAMPLE 6.

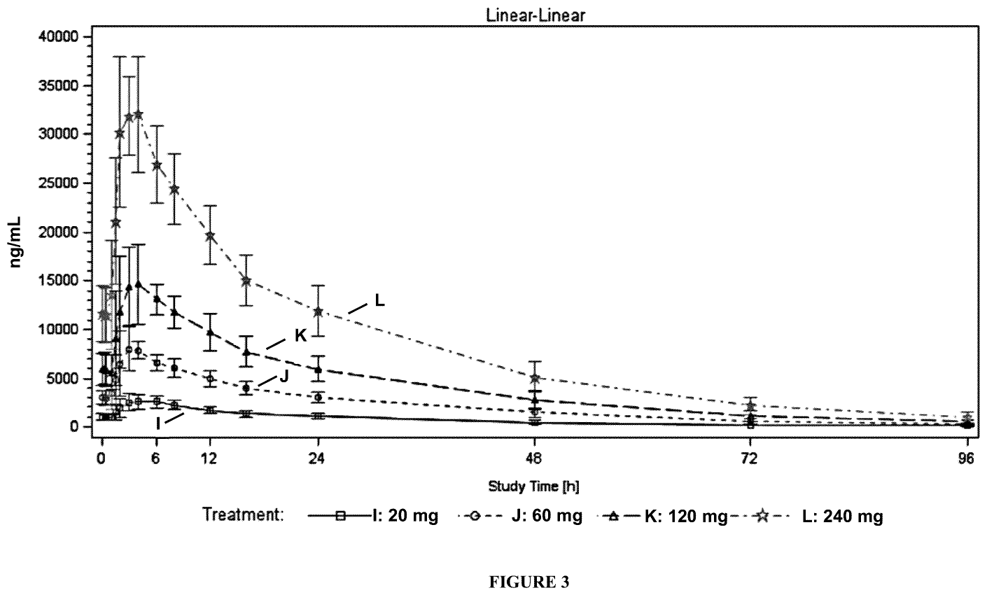

FIG. 3 is a chart depicting the time profile of Compound 1 mean plasma concentration (+/−SD) on Day 14 of Part II of the study detailed in EXAMPLE 6.

### DETAILED DESCRIPTION

The present disclosure provides methods of treating Catecholaminergic Polymorphic Ventricular Tachycardia (CPVT), e.g., CPVT type 1, comprising administering to a subject in need thereof a pharmaceutical composition comprising, in unit dosage form, a ryanodine receptor modulator as described herein, and a pharmaceutically-acceptable excipient. In some embodiments, a compound of the disclosure is ryanodine receptor (RyR) calcium channel stabilizer. In some embodiments, a compound of the disclosure is 4-[(7-methoxy-2,3-dihydro-1,4-benzothiazepin-4(5H)yl)methyl]benzoic acid or a pharmaceutically-acceptable salt thereof, e.g., a hemifumarate salt.

In some embodiments, methods of treating CPVT comprises administering to a subject in need thereof a modified-release pharmaceutical composition comprising 4-[(7-methoxy-2,3-dihydro-1,4-benzothiazepin-4(5H)yl)methyl]benzoic acid or a pharmaceutically-acceptable salt thereof, e.g., a hemifumarate salt, and a pharmaceutically-acceptable excipient.

The compound 4-[(7-methoxy-2,3-dihydro-1,4-benzothiazepin-4(5H)yl)methyl]benzoic acid has the following chemical structure:

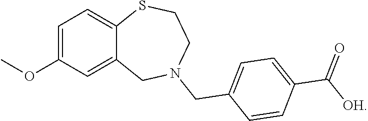 [SMILES: COc1ccc2c(c1)CN(Cc1ccc(C(=O)O)cc1)CCS2]

In some embodiments, 4-[(7-methoxy-2,3-dihydro-1,4-benzothiazepin-4(5H)yl)methyl]benzoic acid is provided in the form of a salt with a pharmaceutically-acceptable acid or base. Non-limiting examples of salts include sodium, potassium, magnesium, hemifumarate, hydrochloride, and hydrobromide salts. In some embodiments, the salt is a sodium salt. In some embodiments, the salt is a hemifumarate salt.

When present as a hemifumarate salt, the compound is herein designated Compound 1. Compound 1 has an empirical formula possessing the following structure or an ionized form thereof:

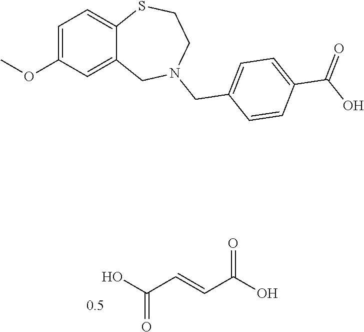 [SMILES: COc1ccc2c(c1)CN(Cc1ccc(C(=O)O)cc1)CCS2 ; O=C(O)/C=C/C(=O)O]

For example, a compound of formula I can be in ionized form, comprising two ionized molecules of 4-[(7-methoxy-2,3-dihydro-1,4-benzothiazepin-4(5H)yl)methyl]benzoic acid.

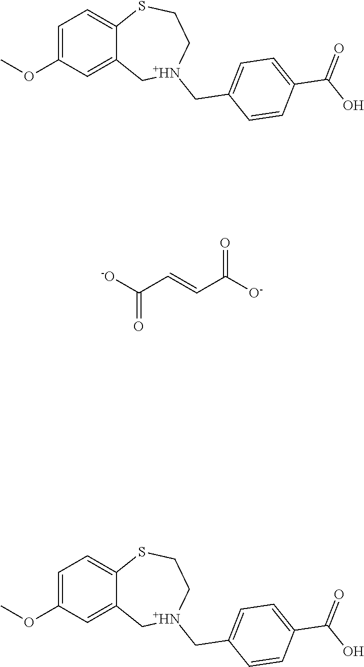

### Ryanodine Receptor 2

In some embodiments, Catecholaminergic Polymorphic Ventricular Tachycardia implicates the Ryanodine Receptor 2 (RyR2) calcium release channel. The RyR2 channel plays a major role in intracellular calcium handling by regulating the release of Ca²⁺ from the sarcoplasmic reticulum (SR) in cardiac myocytes required for ECC in cardiac muscle. The RyR2 channel is a macromolecular complex, which includes four identical RyR2 subunits, each of which binds one Calstabin2 (FKBP12.6), and other interacting proteins such as phosphatases and kinases. Binding of Calstabin2 stabilizes the channel in the closed state during the resting phase of the heart (diastole), thereby preventing diastolic calcium leak from the SR, and functionally couples groups of RyR2 channels to allow synchronous opening during excitation-contraction coupling.

Phosphorylation of RyR2 by protein kinase A (PKA) is an important part of the fight-or-flight response. Phosphorylation increases cardiac EC coupling gain by augmenting the amount of Ca²⁺ released for a given trigger. The process strengthens muscle contraction and improves exercise capacity. This signaling pathway provides a mechanism by which activation of the sympathetic nervous system (SNS), in response to stress, results in increased cardiac output. Phosphorylation of RyR2 by PKA increases the sensitivity of the channel to calcium-dependent activation. The increased sensitivity leads to increased open probability and increased calcium release from the SR into the intracellular cytoplasm.

RyR leak is associated with a variety of cardiac disorders, conditions, and diseases. In some embodiments, the cardiac disorder or disease is catecholaminergic polymorphic ventricular tachycardia (CPVT). In some embodiments, the cardiac disorder or disease is catecholaminergic polymorphic ventricular tachycardia type 1 (CPVT1). In some embodiments, the cardiac disorder or disease is, or is characterized by, an atrial arrhythmia. In some embodiments, the cardiac disorder or disease is, or is characterized by, a ventricular arrhythmia. In some embodiments, the cardiac disorder or disease is, or is characterized by, atrial fibrillation. In some embodiments, the cardiac disorder or disease is, or is characterized by, ventricular fibrillation. In some embodiments, the cardiac disorder or disease is, or is characterized by, atrial tachyarrhythmia. In some embodiments, the cardiac disorder or disease is, or is characterized by, ventricular tachyarrhythmia. In some embodiments, the cardiac disorder or disease is, or is characterized by, atrial tachycardia. In some embodiments, the cardiac disorder or disease is, or is characterized by, ventricular tachycardia. In some embodiments, the cardiac disorder or disease is, or is characterized by, a premature contraction (PC). In some embodiments, the cardiac disorder or disease is, or is characterized by, a premature ventricular contraction (PVC). In some embodiments, the cardiac disorder or disease is, or is characterized by, a bigeminy. In some embodiments, the cardiac disorder or disease is, or is characterized by, sick sinus syndrome. In some embodiments, the cardiac disorder or disease is, or is characterized by, a premature ventricular contraction couplet. In some embodiments, the cardiac disorder or disease is, or is characterized by, sudden cardiac death (SCD). In some embodiments, the cardiac disorder or disease is, or is characterized by, Sudden infant death syndrome (SDIS). In some embodiments, the cardiac disorder or disease is, or is characterized by, sudden unexplained death (SUD).

In some embodiments, the cardiac disorder or disease is characterized by an irregular heartbeat or an arrhythmia. In some embodiments, the cardiac disorder or disease is characterized by an ectopy, such as, for example, ventricular ectopy. In some embodiments, an ectopy (e.g., ventricular ectopy) associated with the cardiac disease or disorder (e.g., CPVT) can be induced by stress, such as catecholaminergic stress. In some embodiments, the ectopy (e.g., ventricular ectopy) is exercise-induced. In some embodiments, the ectopy (e.g., ventricular ectopy) is induced by an elevated heart rate. In some embodiments, the ectopy (e.g., ventricular ectopy) occurs when the subject has an elevated heart rate relative to baseline heart rate of the subject. In some embodiments, the elevated heart rate is a rate at least about 100 beats per minute, at least about 105 beats per minute, at least about 110 beats per minute, at least about 115 beats per minute, at least about 120 beats per minute, at least about 125 beats per minute, at least about 130 beats per minute, at least about 135 beats per minute, at least about 140 beats per minute, at least about 145 beats per minute, at least about 150 beats per minute, at least about 155 beats per minute, at least about 160 beats per minute, at least about 165 beats per minute, at least about 170 beats per minute, at least about 175 beats per minute, at least about 180 beats per minute, at least about 185 beats per minute, at least about 190 beats per minute, at least about 195 beats per minute, or at least about 200 beats per minute. In some embodiments, the elevated heart rate is from about 100 beats per minute to about 200 beats per minute, from about 125 beats per minute to about 200 beats per minute, from about 150 beats per minute to about 200 beats per minute, from about 100 beats per minute to about 125 beats per minute, from about 100 beats per minute to about 150 beats per minute, or from about 100 beats per minute to about 175 beats per minute. In some embodiments, the elevated heart rate is induced by stress. In some embodiments, the stress is catecholaminergic stress. In some embodiments, the elevated heart rate is induced by exercise.

In some embodiments, the present disclosure provides a method of treating catecholaminergic polymorphic ventricular tachycardia (CPVT), comprising administering to a subject in need thereof a therapeutically-effective amount of a ryanodine receptor channel modulator, wherein the treating the catecholaminergic polymorphic ventricular tachycardia (CPVT) reduces a likelihood of developing ectopy in the subject. In some embodiments, ryanodine receptor channel modulator is a compound that is 4-[(7-methoxy-2,3-dihydro-1,4-benzothiazepin-4(5H)yl)methyl]benzoic acid or a pharmaceutically-acceptable salt thereof.

In some embodiments, the present disclosure provides a method of treating catecholaminergic polymorphic ventricular tachycardia (CPVT), comprising administering to a subject in need thereof a therapeutically-effective amount of a ryanodine receptor channel modulator, wherein the treating the catecholaminergic polymorphic ventricular tachycardia (CPVT) reduces a likelihood of atrial fibrillation in the subject. In some embodiments, ryanodine receptor channel modulator is a compound that is 4-[(7-methoxy-2,3-dihydro-1,4-benzothiazepin-4(5H)yl)methyl]benzoic acid or a pharmaceutically-acceptable salt thereof.

### Catecholaminergic Polymorphic Ventricular Tachycardia

Catecholaminergic Polymorphic Ventricular Tachycardia (CPVT) is one of the most lethal inherited arrhythmogenic disorders. CPVT occurs in the absence of structural heart disease and is characterized by adrenergically mediated ventricular arrhythmias associated with a high incidence of Sudden Cardiac Death (SCD). CPVT is a life-threatening disease that is a major cause of unexplained sudden death, particularly in children and young adults. The typical newly diagnosed patient with CPVT is a child or young adult free of structural cardiac disease, with a normal resting electrocardiogram, who presents with stress (e.g., exercise or emotional)-induced palpitations or syncope. If not managed, CPVT is a highly lethal disease with an untreated mortality rate of 30-50% by the age of 40 years.

CPVT is associated with mutations in two genes that code for proteins associated with the sarcoplasmic reticulum (SR) of the cardiomyocyte. The most frequently observed Form Is CPVT type 1 (CPVT1), an autosomal dominant form due to mutations in the RYR2 gene. RYR2 encodes an intracellular SR calcium release channel. CPVT-associated RyR2 mutations result in leaky RyR2 channels that can be associated with decreased binding of the Calstabin2 (FKBP12.6) subunit, which stabilizes the closed state of the channel. Mice heterozygous for the R2474S mutation (which occurs in humans with CPVT1) in RyR2 (RyR2-R2474S mice) can exhibit exercise-induced ventricular arrhythmias and sudden cardiac death.

Under normal physiological conditions, SR Ca²⁺ flux is tightly regulated, and RyR2 interacting proteins contribute to this regulation. For example, Calstabin2 modulates SR Ca²⁺ release by stabilizing the RyR2 closed state. In patients with CPVT1, decreased RyR2-Calstabin2 binding can be associated with RyR2 Ca²⁺ leak.

RyR2-R2474S, a mutant channel harboring a point mutation causing CPVT in humans, adopts a primed state as compared to non-mutated RyR2. The primed state is a transition between the closed and open conformations of these channels. Once primed, the mutant channel is susceptible to transition to the open conformation. Wild type RyR2 channels open during systole, which is the appropriate phase of the cardiac cycle for calcium release. In contrast, the primed, mutant channel can be open in both systole and diastole with exercise or catecholaminergic stress. This inappropriate opening can lead to the ventricular arrythmias characteristic of CPVT. These observations were made under conditions that resemble the channel status during diastole under exercise-induced beta-adrenergic stimulation, e.g., PKA phosphorylation and low calcium concentration.

In some embodiments, the mutant RyR2 protein associated with CPVT is in primed state. In some embodiments, an RyR2 protein in primed state comprises a higher distribution of open probability (Pa) as compared to an RyR2 protein in a resting (closed) state. In some embodiments, an RyR2 protein in primed state is a leaky RyR2 protein, which is characterized by an abnormal Ca′2 leak from the RyR2 channel. In some embodiments, a RyR2 protein in primed state is a leaky RyR2 protein, which is characterized by a higher distribution of open probability (Pₒ) as compared to an RyR2 protein in a resting (closed) state. In some embodiments, a primed state RyR2 comprises about 30% to about 60% of the RyR2 channel in an open state. In some embodiments, a primed state RyR2 comprises about 30%, about 35%, about 40%, about 45%, about 50%, about 55% or about 60% of the RyR channel in an open state.

Ryanodine receptor channel modulators, including Compound 1, offer an innovative approach to the treatment of CPVT1. Ryanodine receptor channel modulators can preferentially bind to leaky RyR2 channels and induce a conformation change that can shift the open probability of the RyR channel towards a closed (resting) state, restore Calstabin 2 binding and repair the channel leak, thereby restoring normal RyR2 function.

In some embodiments, the present disclosure provides a method of treating catecholaminergic polymorphic ventricular tachycardia (CPVT), comprising administering to a subject in need thereof a therapeutically-effective amount of a ryanodine receptor channel modulator. In some embodiments, the present disclosure provides a method of treating catecholaminergic polymorphic ventricular tachycardia (CPVT), comprising administering to a subject in need thereof a therapeutically-effective amount of a compound that is 4-[(7-methoxy-2,3-dihydro-1,4-benzothiazepin-4(5H)yl)methyl]benzoic acid or a pharmaceutically-acceptable salt thereof. In some embodiments, the administering is once daily.

In some embodiments, the present disclosure provides a method of treating catecholaminergic polymorphic ventricular tachycardia (CPVT), comprising administering to a subject in need thereof a therapeutically-effective amount of a ryanodine receptor channel modulator, wherein the treating the catecholaminergic polymorphic ventricular tachycardia (CPVT) reduces a likelihood of sudden cardiac death in the subject. In some embodiments, ryanodine receptor channel modulator is a compound that is 4-[(7-methoxy-2,3-dihydro-1,4-benzothiazepin-4(5H)yl)methyl]benzoic acid or a pharmaceutically-acceptable salt thereof.

In some embodiments, the compound is administered as monotherapy. In some embodiments, the compound is administered in combination with one or more additional therapies. In some embodiments, the compound is administered in combination with a beta-blocker. In some embodiments, the compound is administered in combination with a sodium channel blocker (also referred to herein as a sodium channel inhibitor). In some embodiments, the compound is administered in combination with a beta-blocker and a sodium channel inhibitor.

In some embodiments, the subject is undergoing a treatment regimen for CPVT, wherein the treatment regimen for CPVT comprises a beta-blocker. In some embodiments, the compound is administered in combination with a beta-blocker. In some embodiments, the compound is administered in combination with a beta-blocker, wherein the beta-blocker is administered in an amount that is therapeutically-effective to treat CPVT in the subject in absence of the compound (e.g., 4-[(7-methoxy-2,3-dihydro-1,4-benzothiazepin-4(5H)yl)methyl]benzoic acid or a pharmaceutically-acceptable salt thereof). In some embodiments, the compound is administered in combination with a beta-blocker, wherein the beta-blocker is administered in a reduced amount, wherein the reduced amount is about less than an amount used to treat CPVT in the subject in absence of the compound. In some embodiments, the compound is administered in combination with a beta-blocker, wherein the beta-blocker is administered in a reduced amount, wherein the reduced amount is about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of amount used to treat CPVT in the subject in absence of the compound.

An amount of beta-blocker that is less than an amount that is used to treat CPVT in absence of the compound can be less than a maximum tolerated dose of the beta blocker, which can be, for example, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% less than a maximum tolerated dose of the beta blocker.

An amount of beta-blocker that is less than an amount that is used to treat CPVT in absence of the compound can be an amount that is less than a dose of the beta blocker that is therapeutically effective for CPVT in absence of the compound or pharmaceutically acceptable salt thereof, which can be, for example, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% less than a dose of the beta blocker that is therapeutically effective for CPVT in absence of the compound.

Non-limiting examples of beta-blockers include acebutolol, atenolol, betaxolol, bisoprolol, bucindolol, butaxamine, carteolol, carvedilol, celiprolol, esmolol, labetalol, metoprolol, nadolol, nebivolol, oxprenolol, penbutolol, pindolol, propranolol, sotalol, timolol, and pharmaceutically acceptable salts thereof.

In some embodiments the beta-blocker is a non-selective beta-blocker. A non-selective beta-blocker inhibits both beta-1 receptors located primarily in cardiac muscle, and beta-2 receptors located primarily in the bronchial and vascular musculature. In some embodiments, a non-selective beta-blocker is nadolol, penbutolol, pindolol, propranolol, sotalol, or timolol, or pharmaceutically acceptable-salts thereof. In an embodiment, a non-selective beta-blocker is nadolol or a pharmaceutically-acceptable salt thereof.

In some embodiments, the beta-blocker is a selective beta-blocker. Selective beta blockers (such as metoprolol) can preferentially inhibit beta 1 receptors (cardio-selective). At very high concentrations, this selectivity can be reduced and some beta 2 inhibition can occur. Selectivity is confirmed by the inability to reverse the beta 2-mediated vasodilating effects of epinephrine. This contrasts with the effect of nonselective beta-blockers, which can be capable of reversing the vasodilating effects of epinephrine. In some embodiments, a selective beta-blocker is metoprolol or a pharmaceutically-acceptable salt thereof.

In some embodiments, the subject is undergoing a treatment regimen for CPVT, wherein the treatment regimen for CPVT comprises a sodium channel inhibitor. In some embodiments, the compound is administered in combination with a sodium channel inhibitor. Non-limiting examples of sodium channel inhibitors include flecainide, quinidine, procainamide, disopyramide, lidocaine, mexiletine, tocainide, phenytoin, moricizine, propafenone, lacosamide, rufinamide, fosphenytoin, ethotoin, carbamazepine, eslicarbazepine, pilsicainide, tetrodoxin, aprindine, ajmaline, encainide, propafenone, amiodarone, procainamide, quinidine, oxcarbazepine, moricizine, amiloride, lamotrigine, triamterene, mexiletine, phenytoin, and ranolazine, or a pharmaceutically-acceptable salt thereof. In some embodiments, the sodium channel inhibitor is flecainide or a pharmaceutically-acceptable salt thereof. In some embodiments, the sodium channel inhibitor is flecainide acetate.

In some embodiments, the subject is undergoing a treatment regimen for CPVT, wherein the treatment regimen for CPVT comprises a sodium channel inhibitor. In some embodiments, the compound (e.g., 4-[(7-methoxy-2,3-dihydro-1,4-benzothiazepin-4(5H)yl)methyl]benzoic acid or a pharmaceutically-acceptable salt thereof) is administered in combination with a sodium channel inhibitor. In some embodiments, the compound is administered in combination with a sodium channel inhibitor, wherein the sodium channel inhibitor is administered in an amount that is therapeutically-effective to treat CPVT in the subject in absence of the compound. In some embodiments, the compound is administered in combination with a beta-blocker, wherein the sodium channel inhibitor is administered in a reduced amount, wherein the reduced amount is about less than an amount used to treat CPVT in the subject in absence of the compound. In some embodiments, the compound is administered in combination with a sodium channel inhibitor, wherein the sodium channel inhibitor is administered in a reduced amount, wherein the reduced amount is about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of amount used to treat CPVT in the subject in absence of the compound.

An amount sodium channel inhibitor that is less than an amount that is used to treat CPVT in the absence of the compound can be less than a maximum tolerated dose of the sodium channel inhibitor, which can be, for example, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% less than a maximum tolerated dose of the sodium channel inhibitor.

An amount of sodium channel inhibitor that is less than an amount that is used to treat CPVT in absence of the compound can be an amount that is less than a dose of the sodium channel inhibitor that is therapeutically effective for CPVT in absence of the compound, which can be, for example, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% less than a dose of the sodium channel inhibitor that is therapeutically effective for CPVT in absence of the compound.

In some embodiments, the sodium channel inhibitor is flecainide or a pharmaceutically acceptable salt thereof. An amount of flecainide or pharmaceutically acceptable salt thereof that is less than an amount that is used to treat CPVT in absence of the compound can be an amount that is less than a dose of flecainide or pharmaceutically acceptable salt thereof that is therapeutically effective for CPVT in absence of the compound, which can be, for example, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% less than a dose of flecainide or pharmaceutically acceptable salt that is therapeutically effective for CPVT in absence of the compound.

In some embodiments, left cardiac sympathetic denervation can be used as part of a treatment regimen for CPVT. In some embodiments, a method for treating CPVT comprises use of a beta-blocker in combination with cardiac sympathetic denervation. In some embodiments, a method for treating CPVT comprises use of a sodium channel inhibitor in combination with cardiac sympathetic denervation. In some embodiments, a method for treating CPVT comprises use of a sodium channel inhibitor and a beta-blocker in combination with cardiac sympathetic denervation. In some embodiments, a method for treating CPVT comprises use of the compound (e.g., 4-[(7-methoxy-2,3-dihydro-1,4-benzothiazepin-4(5H)yl)methyl]benzoic acid or a pharmaceutically acceptable salt thereof) in combination with cardiac sympathetic denervation. In some embodiments, a method for treating CVPT comprises use of the compound and a beta-blocker in combination with cardiac sympathetic denervation. In some embodiments, a method for treating CPVT comprises use of the compound and a sodium channel inhibitor in combination cardiac sympathetic denervation. In some embodiments, a method for treating CPVT comprises use of the compound, a sodium channel inhibitor, and a beta-blocker in combination cardiac sympathetic denervation.

In some embodiments, the subject is age 5 years and above. In some embodiments, the subject is an age of about 5 years to about 12 years. In some embodiments, the subject is no more than 5 years, no more than 6 years, no more than 7 years, no more than 8 years, no more than 9 years, no more than 10 years, no more than 11 years, no more than 12 years, no more than 13 years, no more than 14 years, or no more than 15 years. In some embodiments, the subject is at least 5 years, at least 6 years, at least 7 years, at least 8 years, at least 9 years, at least 10 years, at least 11 years, at least 12 years, at least 13 years, at least 14 years, at least 15 years, at least 20 years, at least 25 years, at least 30 years, at least 40 years, at least 50 years, at least 60 years, at least 70 years, at least 80 years, or at least 90 years. In some embodiments, the subject is an age of about 4 years to about 12 years, about 5 years to about 12 years, about 6 years to about 12 years, about 7 years to about 12 years, about 8 years to about 12 years, about 9 years to about 12 years, about 10 years to about 12 years, about 10 years to 16 years, about 12 years to 16 years, about 14 years to about 16 years, about 10 years to about 18 years, about 12 years to about 18 years, about 14 years to about 18 years, or about 16 to about 18 years. In some embodiments, the subject is an age of about 10 to about 120 years, about 11 to about 120 years, about 12 to about 120 years, about 13 to about 120 years, about 14 to about 120 years, about 15 to about 120 years, about 16 to about 120 years, about 17 to about 120 years, or about 18 to about 120 years.

In some embodiments, the compound is formulated to provide a prolonged release of the compound.

In some embodiments, the compound or pharmaceutically acceptable salt thereof is administered to the subject as a pharmaceutical composition in unit dosage form. In some embodiments. In some embodiments, in a study, if the unit dosage form is administered to a study subject, then a prolonged release of the compound or ionized form thereof in the study subject is attained.

In some embodiments, the compound is formulated to provide modified release of the compound (e.g., 4-[(7-methoxy-2,3-dihydro-1,4-benzothiazepin-4(5H)yl)methyl]benzoic acid or a pharmaceutically-acceptable salt thereof). In some embodiments, the compound is formulated to provide a prolonged release of the compound. In some embodiments, the compound is formulated to provide controlled release of the compound. In some embodiments, the compound is formulated to provide extended release of the compound. In some embodiments, the compound is formulated to provide sustained release of the compound. In some embodiments, the compound is formulated to provide delayed release of the compound. In some embodiments, the compound is 4-[(7-methoxy-2,3˜dihydro-1,4-benzothiazepin-4(5H)yl)methyl]benzoic acid hemifumarate.

In some embodiments, the compound (e.g., 4-[(7-methoxy-2,3-dihydro-1,4-benzothiazepin-4(5H)yl)methyl]benzoic acid or a pharmaceutically-acceptable salt thereof) is administered to the subject as a pharmaceutical composition in in unit dosage form. In some embodiments, in a study, if the unit dosage form is administered to a study subject, then the compound or an ionized form thereof is present in the study subject over a period of time, wherein the period of time occurs after administration, wherein the period of time is at least about 12 hours. In some embodiments, in a study, if the unit dosage form is administered to a study subject, then the compound or an ionized form thereof is present in the study subject over a period of time, wherein the period of time occurs after administration, wherein the period of time is at least about 24 hours. Formulations of compounds of the present disclosure (e.g. unit dosage form) are suitable for once-daily administration.

In some embodiments, the present disclosure provides a pharmaceutical composition in unit dosage form comprising a compound that is 4-[(7-methoxy-2,3-dihydro-1,4-benzothiazepin-4(5H)yl)methyl]benzoic acid or a pharmaceutically-acceptable salt thereof. In some embodiments, in a study, if the unit dosage form is administered to a study subject, then the compound an ionized form thereof is present in the study subject over a period of time, wherein the period of time occurs after administration, wherein the period of time is at least about 6 hours. In some embodiments, the period of time is at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours after administration.

In some embodiments, the following pharmacokinetic parameters are used:
AUCₜₐᵤ: The area under the concentration-time curve during a dosing interval (tau) at steady state.
Cₘₐₓ: Maximum observed concentration.
Tₘₐₓ: Time to reach Cₘₐₓ. If the maximum value occurs at more than one time point, Tₘₐₓ is defined as the first time point with this value.
Cₘᵢₙ: Minimum observed concentration.
Tₘᵢₙ: Time to reach Cₘᵢₙ.

In some embodiments, in a study, if the unit dosage form is administered to a study subject, then a maximum plasma concentration (Cₘₐₓ) of the compound (e.g., 4-[(7-methoxy-2,3-dihydro-1,4-benzothiazepin-4(5H)yl)methyl]benzoic acid) or an ionized form thereof is present in the study subject at about 2 to about 6 hours after administration (tₘₐₓ). In some embodiments, in a study, if the unit dosage form is administered to a study subject, then a maximum plasma concentration (Cₘₐₓ) of the compound or an ionized form thereof is present in the study subject at about 2 to about 5 hours after administration (tₘₐₓ). In some embodiments, in a study, if the unit dosage form is administered to a study subject, then a maximum plasma concentration (Cₘₐₓ) of the compound or an ionized form thereof is present in the study subject at about 2 to about 4 hours after administration (tₘₐₓ). In some embodiments, in a study, if the unit dosage form is administered to a study subject, then a maximum plasma concentration (Cₘₐₓ) of the compound or an ionized form thereof is present in the study subject at about 3 to about 4 hours after administration (tₘₐₓ). In some embodiments, in a study, if the unit dosage form is administered to a study subject, then the maximum plasma concentration of the compound or an ionized form thereof in the study subject is reached about 3 hours after administration. In some embodiments, in a study, if the unit dosage form is administered to a study subject, then the maximum plasma concentration of the compound or an ionized form thereof in the study subject is reached about 3.5 hours after administration. In some embodiments, in a study, if the unit dosage form is administered to a study subject, then the maximum plasma concentration of the compound or an ionized form thereof in the study subject is reached about 4 hours after administration. In some embodiments, in a study, if the unit dosage form is administered to a study subject, then the maximum plasma concentration of the compound or an ionized form thereof in the study subject is reached about 4.5 hours after administration. In some embodiments, in a study, if the unit dosage form is administered to a study subject, then the maximum plasma concentration of the compound or an ionized form thereof in the study subject is reached about 5 hours after administration. In some embodiments, in a study, if the unit dosage form is administered to a study subject, then the maximum plasma concentration of the compound or an ionized form thereof in the study subject is reached about 5.5 hours after administration. In some embodiments, in a study, if the unit dosage form is administered to a study subject, then the maximum plasma concentration of the compound or an ionized form thereof in the study subject is reached about 6 hours after administration. In some embodiments, in a study, if the unit dosage form is administered to a study subject, then the maximum plasma concentration of the compound or an ionized form thereof in the study subject is reached about 2˜10 hours, about 2-9 hours, about 2-8 hours, about 2-7 hours, about 1-10 hours, about 1-9 hours, about 1-8 hours, about 1-7 hours, about 1-6 hours, about 1-5 hours, about 1-4 hours, about 1-3 hours, or about 1-2 hours after administration.

In some embodiments, in a study, if the unit dosage form is administered to a study subject, then an in-vivo half-life of the compound (e.g., 4˜ [(7-methoxy-2,3-dihydro-1,4-benzothiazepin-4(5H)yl)methyl]benzoic acid) or an ionized form thereof of about 14 to about 21 hours is obtained in the study subject. In some embodiments, the in-vivo half-life of 4-[(7-methoxy-2,3-dihydro-1,4-benzothiazepin-4(5H)yl)methyl]benzoic acid or an ionized form thereof is about 14 hours. In some embodiments, the in-vivo half-life of 4-[(7-methoxy-2,3-dihydro-1,4-benzothiazepin-4(5H)yl)methyl]benzoic acid or an ionized form thereof is about 15 hours. In some embodiments, the in-vivo half-life of 4-[(7-methoxy-2,3-dihydro-1,4-benzothiazepin-4(5H)yl)methyl]benzoic acid or an ionized form thereof is about 16 hours. In some embodiments, the in-vivo half-life of 4-[(7-methoxy-2,3-dihydro-1,4-benzothiazepin-4(5H)yl)methyl]benzoic acid or an ionized form thereof is about 17 hours. In some embodiments, the in-vivo half-life of 4-[(7-methoxy-2,3-dihydro-1,4-benzothiazepin-4(5H)yl)methyl]benzoic acid or an ionized form thereof is about 18 hours. In some embodiments, the in-vivo half-life of 4-[(7-methoxy-2,3-dihydro-1,4-benzothiazepin-4(5H)yl)methyl]benzoic acid or an ionized form thereof is about 19 hours. In some embodiments, the in-vivo half-life of 4-[(7-methoxy-2,3-dihydro-1,4-benzothiazepin-4(5H)yl)methyl]benzoic acid or an ionized form thereof is about 20 hours. In some embodiments, the in-vivo half-life of 4˜ [(7-methoxy-2,3-dihydro-1,4-benzothiazepin-4(5H)yl)methyl]benzoic acid or an ionized form thereof is about 21 hours.

In some embodiments, the in-vivo half-life is about 12-24 hours, about 14-24 hours, about 16-24 hours, about 18-24 hours, about 20-24 hours, about 12-22 hours, about 14-22 hours, about 16-22 hours, about 18-22 hours, or about 20-22 hours. In some embodiments, the pharmaceutically-acceptable salt is a hemifumarate salt.

In some embodiments, half-life (t_{1/2,z}, or t½) is the terminal elimination half-life of the compound, calculated as:

$${t}_{1 / 2 , z} = ln ( 2 ) / {λ}_{z} ( {λ}_{z} being the rate constant of the terminal phase ) .$$

In some embodiments, the present disclosure provides a pharmaceutical composition in unit dosage form comprising a compound that is 4-[(7-methoxy-2,3-dihydro-1,4-benzothiazepin-4(5H)yl)methyl]benzoic acid or a pharmaceutically-acceptable salt thereof, wherein in a study, if the unit dosage form is administered to a study subject, then an accumulation ratio for Cₘₐₓ of the compound (e.g., 4˜ [(7-methoxy-2,3-dihydro-1,4-benzothiazepin-4(5H)yl)methyl]benzoic acid) or an ionized form thereof from about 1.4 and about 1.8 is observed in the study subject, wherein said accumulation ratio is calculated as a ratio of Coax on Day 28/Cox on Day 1, wherein Cₘₐₓ is maximum observed plasma concentration. In some embodiments, the accumulation ratio Cₘₐₓ from about 1.4 to about 1.5, from about 1.4 to about 1.6, from about 1.4 to about 1.7, from about 1.5 to about 1.7, from about 1.5 to about 1.8, from about 1.6 to about 1.7, or from about 1.7 to about 1.8. In some embodiments, the pharmaceutically-acceptable salt is a hemifumarate salt.

In some embodiments, the present disclosure provides a pharmaceutical composition in unit dosage form comprising a compound that is 4-[(7-methoxy-2,3-dihydro-1,4-benzothiazepin-4(5H)yl)methyl]benzoic acid or a pharmaceutically-acceptable salt thereof, wherein in a study, if the unit dosage form is administered to a study subject, then an AUC of the compound or an ionized form thereof from about 1.4 to about 1.8 is present in the study subject. For example, the accumulation ratio AUC can be from about 1.4 to about 1.5, from about 1.4 to about 1.6, from about 1.4 to about 1.7, from about 1.5 to about 1.7, from about 1.5 to about 1.8, from about 1.6 to about 1.7, or from about 1.7 to about 1.8. In some embodiments, the pharmaceutically-acceptable salt is a hemifumarate salt.

The accumulation ratio for AUC is calculated as a ratio of AUCₜₐᵤ on Day 28/AUC₀₋₂₄ Day 1,
wherein:
   AUC is area under the concentration-time curve;
   AUCₜₐᵤ is area under the concentration-time curve during a dosing interval (tau) at steady state; and
   AUC₀₋₂₄ is area under the concentration-time curve, from time 0 to 24 hours post-dose.

In some embodiments, the present disclosure provides a pharmaceutical composition in unit dosage form comprising a compound that is 4-[(7-methoxy-2,3-dihydro-1,4-benzothiazepin-4(5H)yl)methyl]benzoic acid or a pharmaceutically-acceptable salt thereof, wherein in a study, if the unit dosage form is administered to a study subject, then an maximum observed plasma concentration of the compound or an ionized form thereof of no more than about 35 μg/mL of 4-[(7-methoxy-2,3-dihydro-1,4-benzothiazepin-4(5H)yl)methyl]benzoic acid or an ionized form thereof is present in the study subject. In some embodiments, a maximum observed plasma concentration of no more than about 10 μg/mL, no more than about 15 μg/mL, no more than about 20 g/mL, no more than about 25 g/mL, no more than about 30 μg/mL, no more than about 35 g/mL, no more than about 40 ng/ml., no more than about 45 μg/ml, or no more than about 50 μg/mL of 4-[(7-methoxy-2,3-dihydro-1,4-benzothiazepin-4(5H)ylmethyl]benzoic acid or an ionized form thereof is present in the study subject. In some embodiments, a maximum observed plasma concentration of about 10 μg/mL, about 15 μg/mL about 20 μg/mL, about 25 μg/mL, about 30 μg/mL, or about 35 μg/mL of 4-[(7-methoxy-2,3-dihydro-1,4-benzothiazepin-4(5H)yl)methyl]benzoic acid or an ionized form thereof is present in the study subject. In some embodiments, the pharmaceutically-acceptable salt is a hemifumarate salt.

In some embodiments, the present disclosure provides a pharmaceutical composition in unit dosage form comprising a compound that is 4-[(7-methoxy-2,3-dihydro-1,4-benzothiazepin-4(5H)yl)methyl]benzoic acid or a pharmaceutically-acceptable salt thereof, wherein in a study, if the unit dosage form is administered to a study subject, then a steady-state plasma concentration of the compound or an ionized form thereof occurs in the study subject in a range of about 3 to about 7 days after initial administration. In some embodiments, steady-state is reached after about 3 days, about 4 days, about 5 days, about 6 days, or about 7 days of once-daily administrations. In some embodiments, a steady-state plasma concentration of the compound or ionized form thereof occurs in the study subject in a range of about 2 to about 10 days, about 2 to about 9 days, about 2 to about 8 days, about 2 to about 7 days, about 2 to about 6 days, about 2 to about 5 days, about 2 to about 4 days, about 2 to about 3 days, about 3 to about 10 days, about 3 to about 9 days, about 3 to about 8 days, about 3 to about 6 days, about 3 to about 5 days, about 3 to about 4 days, about 4 to about 10 days, about 4 to about 9 days, about 4 to about 8 days, about 4 to about 7 days, about 4 to about 6 days, or about 4 to about 5 days after initial administration. In some embodiments, the pharmaceutically-acceptable salt is a hemifumarate salt.

Pharmacokinetic parameters (e.g., AUCₜₐᵤ, AUC₀₋₂₄, Cₘₐₓ, Tₘₐₓ, Cₘᵢₙ, Tₘᵢₙ, t_{1/2}) can be measured in a study. In some embodiments, the study comprises administering the pharmaceutical composition in unit dosage form to the study subject, and after the administering, collecting a blood sample from the study subject and measuring a plasma concentration of 4-[(7-methoxy-2,3-dihydro-1,4-benzothiazepin-4(5H)yl)methyl]benzoic acid or an ionized form thereof in the blood sample.

In some embodiments, the study subject is a male. In some embodiments, the study subject is a female. In some embodiments, the study subject is afflicted with Catecholaminergic Polymorphic Ventricular Tachycardia (CPVT). In some embodiments, the study subject is afflicted with Catecholaminergic Polymorphic Ventricular Tachycardia type 1 (CPVT1). In some embodiments, the study subject is not afflicted by CPVT. In some embodiments, a study subject that is used to assess pharmacokinetic and/or pharmacodynamic parameters of a tablet herein is not afflicted with Catecholaminergic Polymorphic Ventricular Tachycardia (CPVT).

In some embodiments, the unit dosage form is a gastro-resistant tablet.

In some embodiments, the pharmaceutical composition in unit dosage form is administered to the study subject in daily doses. In some embodiments, the pharmaceutical composition in unit dosage form is administered to the study subject one time per day. In some embodiments, the pharmaceutical composition in unit dosage form is administered to the study subject two times per day. In some embodiments, the pharmaceutical composition in unit dosage form is administered to the study subject three times per day.

In some embodiments, the pharmaceutical composition in unit dosage form is administered to the study subject on an at least daily basis for 14 days. In some embodiments, the pharmaceutical composition in unit dosage form is administered to the study subject on an at least daily basis for about 28 days. In some embodiments, the pharmaceutical composition in unit dosage form is administered to the study subject on an at least daily basis for about a month. In some embodiments, the pharmaceutical composition in unit dosage form is administered to the study subject on an at least daily basis for about 2 months, 3 months, 4 months, five months, six months, seven months, eight months, nine months, ten months, eleven months twelve months (1 year), 2 years, 3 years, 4 years, 5 years, 6 years, 7 years, 8 years, 9 years, 10 years or longer. In some embodiments, the pharmaceutical composition in unit dosage form is administered to the study subject daily for the life of the subject.

In some embodiments, the pharmaceutical composition in unit dosage form comprises 4-[(7-methoxy-2,3-dihydro-1,4-benzothiazepin-4(5H)yl)methyl]benzoic acid hemifumarate.

In some embodiments, the pharmaceutical composition in unit dosage form comprises an amount of 4-[(7-methoxy-2,3-dihydro-1,4-benzothiazepin-4(5H)yl)methyl]benzoic acid hemifumarate that is equivalent to about 20 mg of 4-[(7-methoxy-2,3-dihydro-1,4-benzothiazepin-4(5H)yl)methyl]benzoic acid. In some embodiments, the pharmaceutical composition in unit dosage form comprises an amount of 4-[(7-methoxy-2,3-dihydro-1,4-benzothiazepin-4(5H)yl)methyl]benzoic acid hemifumarate that is equivalent to about 50 mg of 4-[(7-methoxy-2,3-dihydro-1,4-benzothiazepin-4(5H)yl)methyl]benzoic acid. In some embodiments, the pharmaceutical composition in unit dosage form comprises an amount of 4-[(7-methoxy-2,3-dihydro-1,4-benzothiazepin-4(5H)yl)methyl]benzoic acid hemifumarate that is equivalent to about 100 mg of 4-[(7-methoxy-2,3˜dihydro-1,4-benzothiazepin-4(5H)yl)methyl]benzoic acid. In some embodiments, the pharmaceutical composition in unit dosage form comprises an amount of 4-[(7-methoxy-2,3-dihydro-1,4-benzothiazepin-4(5H)yl)methyl]benzoic acid hemifumarate that is equivalent to about 40 mg, about 50 mg, about 80 mg, about 100 mg, about 160 mg, about 240 mg, or about 400 mg of 4-[(7-methoxy-2,3-dihydro-1,4-benzothiazepin-4(5H)yl)methyl]benzoic acid.

In some embodiments, the pharmaceutical composition is administered to a study subject in a clinical study. In some embodiments, the clinical study is a controlled study. In some embodiments, the clinical study is a blinded study. In some embodiments, a clinical study is a double-blinded study. In some embodiments, the clinical study is a double-blinded, placebo-controlled study.

In some embodiments, the study is a placebo-controlled study. In some embodiments, the controlled study further comprises administering a placebo unit dosage form (e.g., a placebo tablet) to a control subject. In some embodiments, a clinical study includes administering 4˜ [(7-methoxy-2,3-dihydro-1,4-benzothiazepin-4(5H)yl)methyl]benzoic acid or a pharmaceutically-acceptable salt thereof for a first period, then administering placebo for a second period. In some embodiments, the clinical study comprises administering placebo for a first period, then administering 4-[(7-methoxy-2,3-dihydro-1,4-benzothiazepin-4(5H)yl)methyl]benzoic acid or a pharmaceutically-acceptable salt for a second period. In some embodiments, the second period begins at the end of the first period. In some embodiments, the first period is followed by a wash-out period where the subject receives no therapy, prior to the beginning of the second period. The wash-out period can be, for example, 1 day, 2 days, 3 days, 4 days, 5 days, 6 days, 7 days, 8 days, 9 days, 10 days, 11 days, 12 days, 13 days, 14 days, or longer.

In some embodiments, a clinical study includes measuring a plasma concentration of 4-[(7-methoxy-2,3-dihydro-1,4-benzothiazepin-4(5H)yl)methyl]benzoic acid or an ionized form thereof in the study subject. In some embodiments, measuring a plasma concentration comprises collecting a blood sample from the study subject and measuring the plasma concentration of 4-[(7-methoxy-2,3-dihydro-1,4-benzothiazepin-4(5H)yl)methyl]benzoic acid or an ionized form thereof in the blood sample. In some embodiments, the blood samples are collected from the study subject before the administering. In some embodiments, the blood samples are collected from the study subject about 0.25, about 0.5, about 1, about 1.5, about 2, about 3, about 4, about 5, about 6, about 8, about 10, about 12, about 16, about 24, about 36, about 48, about 60, about 72, or about 96 hours after the administering. In some embodiments, the blood samples are collected from the study subject about 0.25, about 0.5, about 1, about 1.5, about 2, about 3, about 4, about 5, about 6, about 8, about 10, about 12, about 16, about 24, about 36, about 48, about 60, about 72, and about 96 hours after the administering. In some embodiments, the blood samples are collected from the study subject on every day of a controlled study. In some embodiments, the blood samples are collected from the study subject before the administering, on the first day of the controlled study, on the mid-point of the controlled study, and on the last day of the controlled study. In some embodiments, the blood samples are collected from the study subject on the first day of the controlled study, on the mid-point of the controlled study, and on the last day of the controlled study.

In some embodiments, safety and tolerability of the pharmaceutical composition is determined by monitoring adverse events over the duration of the study. In some embodiments, the safety and tolerability of the pharmaceutical composition is determined by patient interviews, patient diary reviews, physical examinations, electrocardiograms, cardiac monitoring, vital signs, or laboratory safety tests.

In some embodiments, the controlled study comprises:
(a) administering the pharmaceutical composition in unit dosage form to the study subject;
(b) after the administering, collecting blood samples from the study subject about 0.25, about 0.5, about 1, about 1.5, about 2, about 3, about 4, about 5, about 6, about 8, about 10, about 12, about 16, about 24, about 36, about 48, about 60, about 72, and about 96 hours after the administering;
(c) measuring a plasma concentration of 4-[(7-methoxy-2,3-dihydro-1,4-benzothiazepin-4(5H)yl)methyl]benzoic acid or an ionized form thereof in the blood samples,
(d) administering a placebo to a control subject;
(e) after the administering the placebo, collecting control blood samples from the control subject about 0.25, about 0.5, about 1, about 1.5, about 2, about 3, about 4, about 5, about 6, about 8, about 10, about 12, about 16, about 24, about 36, about 48, about 60, about 72, and about 96 hours after the administering the placebo; and
(f) measuring a plasma concentration of 4-[(7-methoxy-2,3-dihydro-1,4-benzothiazepin-4(5H)yl)methyl]benzoic acid or an ionized form thereof in the control blood samples,
   wherein:
the pharmaceutical composition in unit dosage form comprises an amount of 4-[(7-methoxy-2,3-dihydro-1,4-benzothiazepin-4(5H)yl)methyl]benzoic acid hemifumarate that is equivalent to about 40 mg, about 80 mg, about 160 mg, about 240 mg, or about 400 mg of 4-[(7-methoxy-2,3-dihydro-1,4-benzothiazepin-4(5H)yl)methyl]benzoic acid;
the placebo, relative to the pharmaceutical composition in unit dosage form, is an analogous dosage form that lacks 4-[(7-methoxy-2,3-dihydro-1,4-benzothiazepin-4(5H)yl)methyl]benzoic acid hemifumarate; and
the control subject and the study subject are healthy males.

In some embodiments, the healthy males are males who are not afflicted with CPVT.

In some embodiments, the pharmaceutical composition in unit dosage form administered to the study subject is one or more of a tablet according to TABLE 1. In some embodiments, the pharmaceutical composition in unit dosage form administered to the study subject is 1, 2, 3, 4, 5, 6, 7, 8, 9, or 10 of a tablet according to TABLE 1. In some embodiments, the pharmaceutical composition in unit dosage form administered to the study subject is one or more of a tablet according to TABLE 6. In some embodiments, the pharmaceutical composition in unit dosage form administered to the study subject is 1, 2, 3, 4, 5, 6, 7, 8, 9, or 10 of a tablet according to TABLE 6.

In some embodiments, the placebo is an enteric-coated tablet according to TABLE 2. In some embodiments, the placebo, relative to the pharmaceutical composition in unit dosage form, is an analogous dosage form that lacks 4-[(7-methoxy-2,3-dihydro-1,4-benzothiazepin-4(5H)yl)methyl]benzoic acid hemifumarate.

In some embodiments, the unit dosage form is a tablet, the tablet comprising a core, a sub-coating layer substantially covering the core, and a coating layer substantially covering the sub-coating layer, wherein
the core comprises 4-[(7-methoxy-2,3-dihydro-1,4-benzothiazepin-4(5H)yl)methyl]benzoic acid hemifumarate, mannitol, microcrystalline cellulose, croscarmellose sodium, magnesium stearate, maltodextrin, colloidal anhydrous silica, and sodium stearyl fumarate;
the sub-coating layer comprises hypromellose, microcrystalline cellulose, and stearic acid; and
the coating layer comprises hypromellose acetate succinate, triethyl citrate, sodium lauryl sulfate, and talc.

### Pharmaceutically-Acceptable Salts

The disclosure provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, or pyrazine.

In some embodiments, an ammonium salt is a triethyl amine salt, a trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, trifluoroacetic acid, mandelic acid, cinnamic acid, aspartic acid, stearic acid, palmitic acid, glycolic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a trifluoroacetate salt, a mandelate salt, a cinnamate salt, an aspartate salt, a stearate salt, a palmitate salt, a glycolate salt, a propionate salt, a butyrate salt, a fumarate salt, a hemifumarate salt, a succinate salt, a methanesulfonate salt, an ethanesulfonate salt, a benzenesulfonate salt, a p-toluenesulfonate salt, a citrate salt, an oxalate salt, or a maleate salt.

### Gastro-Resistant Formulations

In some embodiments, a modified-release formulation is a gastro-resistant formulation. In some embodiments, a modified-release formulation is a gastro-resistant formulation in unit dosage form. In some embodiments, a modified-release formulation is a gastro-resistant formulation in a unit solid dosage form.

In some embodiments, a delayed-release formulation is a gastro-resistant formulation. In some embodiments, a delayed-release formulation is a gastro-resistant formulation in unit dosage form. In some embodiments, a delayed-release formulation is a gastro-resistant formulation in a unit solid dosage form.

Gastro-resistant tablets are delayed-release tablets that can resist acidic gastric fluid and release their active substance(s) in the intestinal fluid. Gastro-resistant tablets can be prepared from granules or particles already covered with a gastro-resistant coating or alternatively by covering tablets with a gastro-resistant coating (enteric-coated tablets). The pH range of fluids in various segments of the gastrointestinal tract provide environmental stimuli for responsive drug release.

In some embodiments, the present disclosure provides a gastro-resistant tablet comprising a tablet core, the tablet core comprising a therapeutically-effective amount of 4-[(7-methoxy-2,3-dihydro-1,4-benzothiazepin-4(5H)yl)methyl]benzoic acid or a pharmaceutically-acceptable salt thereof as an active ingredient, a subcoating layer substantially surrounding the core, and an enteric coating, the enteric coating comprising an enteric polymer, the enteric coating substantially surrounding the subcoat.

In some embodiments, the present disclosure provides a gastro-resistant tablet comprising a tablet core, the tablet core comprising a therapeutically-effective amount of 4-[(7-methoxy-2,3-dihydro-1,4-benzothiazepin-4(5H)yl)methyl]benzoic acid hemifumarate as an active ingredient, a subcoating layer substantially surrounding the core, and an enteric coating, the enteric coating comprising an enteric polymer, the enteric coating substantially surrounding the subcoat.

In some embodiments, the present disclosure provides a tablet comprising a core, a sub-coating layer substantially covering the core, and a coating layer substantially covering the sub-coating layer, wherein
the core comprises mannitol, microcrystalline cellulose, croscarmellose sodium, magnesium stearate, maltodextrin, colloidal anhydrous silica, sodium stearyl fumarate, and an amount of 4-[(7-methoxy-2,3-dihydro-1,4-benzothiazepin-4(5H)yl)methyl]benzoic acid hemifumarate that is equivalent to 20 mg of 4-[(7-methoxy-2,3-dihydro-1,4-benzothiazepin-4(5H)yl)methyl]benzoic acid;
the sub-coating layer comprises hypromellose, microcrystalline cellulose, and stearic acid; and
the coating layer comprises hypromellose acetate succinate, triethyl citrate, sodium lauryl sulfate, and talc.

In some embodiments, the present disclosure provides a tablet comprising a core, a sub-coating layer substantially covering the core, and a coating layer substantially covering the sub-coating layer, wherein
the core comprises mannitol, microcrystalline cellulose, croscarmellose sodium, magnesium stearate, maltodextrin, colloidal anhydrous silica, sodium stearyl fumarate, and an amount of 4-[(7-methoxy-2,3-dihydro-1,4-benzothiazepin-4(5H)yl)methyl]benzoic acid hemifumarate that is equivalent to 50 mg of 4-[(7-methoxy-2,3-dihydro-1,4-benzothiazepin-4(5H)yl)methyl]benzoic acid;
the sub-coating layer comprises hypromellose, microcrystalline cellulose, and stearic acid; and
the coating layer comprises hypromellose acetate succinate, triethyl citrate, sodium lauryl sulfate, and talc.

In some embodiments, the present disclosure provides a tablet comprising a core, a sub-coating layer substantially covering the core, and a coating layer substantially covering the sub-coating layer, wherein
the core comprises mannitol, microcrystalline cellulose, croscarmellose sodium, magnesium stearate, maltodextrin, colloidal anhydrous silica, sodium stearyl fumarate, and an amount of 4-[(7-methoxy-2,3-dihydro-1,4-benzothiazepin-4(5H)yl)methyl]benzoic acid hemifumarate that is equivalent to 100 mg of 4-[(7-methoxy-2,3-dihydro-1,4-benzothiazepin-4(5H)yl)methyl]benzoic acid;
the sub-coating layer comprises hypromellose, microcrystalline cellulose, and stearic acid; and
the coating layer comprises hypromellose acetate succinate, triethyl citrate, sodium lauryl sulfate, and talc.

In some embodiments, enteric-coated gastro-resistant tablets are composed of three layers: (1) a drug-containing core tablet (immediate release function); (2) a subcoat layer substantially covering the core, which subcoat layer can include a swellable, hydrophobic polymer layer (e.g., hydroxypropyl cellulose (HPC) or hydroxypropylmethyl cellulose (HPMC) (time release function)); and (3) an enteric coating layer comprising an enteric polymer, the enteric coating layer substantially covering the subcoat layer (acid resistance function). The tablet does not substantially release the drug in the stomach due to the acid resistance of the outer enteric coating layer. The enteric coating layer rapidly dissolves after gastric emptying and the intestinal fluid begins to erode the subcoat polymer layer. Rapid drug release occurs after the erosion front reaches the core tablet after gastric emptying. The time needed for the core tablet to become accessible by dissolution of the eroding layers is the lag phase, the duration of which is controlled either by the mass or composition of the polymer in the subcoat layer.

In some embodiments, a gastro-resistant formulation is a delayed-release formulation due to, e.g., sensitivity to pH resulting from an enteric coating, and a modified-release formulation due to, e.g., the presence of a polymer in the subcoat layer. In some embodiments, a formulation is characterized by a delayed-release profile such that all or substantially all of the formulation transits the stomach and is released in the small intestine. In addition, due to the presence of a polymer in the subcoating layer, slow erosion of the formulation (lag phase) results in prolonged-release of the active ingredient relative to an immediate release formulation.

In some embodiments, the gastro-resistant formulation is resistant to disintegration in gastric fluid. For example, in some embodiments, less than about 10%, less than about 9%, less than about 8%, less than about 7%, less than about 6%, less than about 5%, less than about 4%, less than about 3%, less than about 2%, or less than about 1% of the active ingredient is released from the formulation in gastric fluid or at a pH mimicking that of gastric fluid. A pH of gastric fluid varies with the presence or absence of food, and typically ranges from about 1.5 to about 3.5. In some embodiments, the gastro-resistant formulation does not substantially disintegrate for at least about 15 minutes after exposure to gastric fluid. For example, the gastro-resistant formulation does not substantially disintegrate for at least about 30 minutes, or at least about 45 minutes, or at least about 60 minutes, or at least about 75 minutes, or at least about 90 minutes, or at least about 120 minutes, or at least about 180 minutes, or even longer after exposure to gastric fluid. In some embodiments, the gastro-resistant formulation is resistant to disintegration in gastric fluid in the absence of food. In some embodiments, the gastro-resistant formulation is resistant to disintegration in gastric fluid in the presence of food.

In some embodiments, the gastro-resistant formulation (e.g., a gastro-resistant tablet) does not substantially disintegrate at a pH up to 5,5. For example, the gastro-resistant formulation (e.g., gastro-resistant tablet) releases less than about 10% of an active ingredient at a pH up to 5.5. For example, the gastro-resistant formulation (e.g., gastro-resistant tablet) releases less than about 9%, less than about 8%, less than about 7%, less than about 6%, less than about 5%, less than about 4%, less than about 3%, less than about 2%, or less than about 1% of an active ingredient at a pH up to about 5.5.

In some embodiments, the gastro-resistant formulation does not substantially disintegrate after exposure to a pH up to about 5.5, e.g., a pH up to 4.5, up to 4, up to 3.5, up to 3, up to 2.5, up to 2, or lower. In some embodiments, the gastro-resistant formulation does not substantially disintegrate for at least about 15 minutes after exposure to a pH up to about 5.5. For example, the gastro-resistant formulation does not substantially disintegrate for at least about 30 minutes, or at least about 45 minutes, or at least about 60 minutes, or at least about 75 minutes, or at least about 90 minutes, or at least about 120 minutes, or at least about 180 minutes, or even longer after exposure to a pH up to about 5.5.

In some embodiments, the gastro-resistant formulation (e.g., gastro-resistant tablet) substantially disintegrates at neutral pH (pH=7), or a pH that is close to neutral, e.g., a pH 6.8, or higher. In some embodiments, delayed-release formulations release at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99% at a pH about 6.8 or higher. Such release can occur rapidly, e.g., within 30 minutes, 40 minutes, 50 minutes, or 60 minutes, or 120 minutes, or 180 minutes after the enteric layer and/or the subcoat layer are eroded and the drug-containing core is exposed.

In some embodiments, the gastro-resistant formulation (e.g., tablet) comprises an enteric coating layer. Enteric coated tablets are solid, oral unit dosage forms that are designed to pass through the stomach and release the drug in the small intestine. In some embodiments, enteric coatings prevent release of the active ingredient before the tablet reaches the small intestine. Once the formulation reaches the small intestine, the enteric coating dissolves and the active ingredient is released. Release of the active ingredient can be according to an immediate release profile, e.g., at least about 70%, at least about 75%, at least about 80%, at least about 85%, at least about 90%, at least about 95%, at least about 95%, at least about 96%, at least about 97%, at least about 98%, or at least about 99% of the active ingredient is released within 1 hour of reaching the small intestine.

In some embodiments, disintegration is determined by measuring dissolution of a gastro-resistant formulation in a medium having a pH at or below 5.5. In some embodiments, disintegration is determined by measuring dissolution of a gastro-resistant formulation in a medium having a pH between about 1.0 and about 2.0, between about 2.0 and about 3.0, between about 3.0 and about 4.0, between about 4.0 and 5.0, or between about 4.5 and 5.5. In some embodiments, the medium having a pH at or below 5.5 is a HCl solution having a pH of about 1.0. In some embodiments, the medium having a pH at or below 5.5 is a HCl solution having a pH of about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0, about 5.2, about 5.4, or about 5.5. In some embodiments, the medium having a pH up to 5.5 is a 0.1N HCl solution having a pH of about 1.0. In some embodiments, the medium having a pH up to 5.5 is a 0.1N HCl solution having a pH of about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0, about 5.2, about 5.4, or about 5.5.

In some embodiments, disintegration is determined by measuring dissolution of a gastro-resistant formulation in a medium having a pH of about 6.0 to about 7.0. In some embodiments, disintegration is determined by measuring dissolution of a gastro-resistant formulation in a medium having a pH of about 6.2 to about 7.0, about 6.4 to about 7.0, about 6.6 to about 7.0, about 6.8 to about 7.0, about 6.0, about 6.2, about 6.4, about 6.6, about 6.8, or about 7.0. In some embodiments, the medium comprises a phosphate buffer.

In some embodiments, an enteric coating layer rapidly dissolves after gastric emptying and the intestinal fluid begins to erode the subcoat polymer layer. Rapid drug release occurs after the erosion front reaches the core tablet after gastric emptying. The time needed for the core tablet to become accessible by dissolution of the eroding layers is the lag phase. In some embodiments, duration of the lag phase is controlled by varying the mass of the polymer in the subcoat layer. In some embodiments, duration of the lag phase is controlled by varying the nature of the polymer in the subcoat layer. In some embodiments, the duration of the lag phase is controlled by varying the mass and composition of the polymer in the subcoat layer. In some embodiments, the polymer is hydroxypropylcellulose (HPC). In some embodiments, the polymer is hydroxypropylmethyl cellulose (HPMC).

In some embodiments, the present disclosure provides a method of treating catecholaminergic polymorphic ventricular tachycardia (CPVT), comprising administering to a subject in need thereof the gastro-resistant formulation (e.g., gastro-resistant tablet).

In some embodiments, a gastro-resistant pharmaceutical composition of the present disclosure is administered to the subject in a fed state (for example, during a meal or within at most about 1 hour, at most about 2 hours, at most about 3 hours, at most about 4 hours, at most about 5 hours, at most about 6 hours, or at most about 7 hours after a meal). In some embodiments, a gastro-resistant pharmaceutical composition of the present disclosure is administered to the subject during a meal or about 1 hour, about 2 hours, about 3 hours, about 4 hours, about 5 hours, about 6 hours, or about 7 hours after a meal. In some embodiments, a gastro-resistant pharmaceutical composition of the present disclosure is administered to the subject in a fasted state (for example, more than 7 hours, at least 8 about hours, at least about 9 hours, at least about 10 hours, at least about 11 hours, or at least about 12 hours after a meal, or longer). In some embodiments, a gastro-resistant pharmaceutical composition of the present disclosure is administered to the subject during a meal or about 7 hours, about 8 hours, about 9 hours, about 10 hours, about 11 hours, about 12 hours, about 14 hours, about 16 hours, about 18 hours, or about 24 hours after a meal. In some embodiments, a meal is a high fat meal. In some embodiments, a meal is a low fat meal.

In some embodiments, the gastro-resistant pharmaceutical composition of the present disclosure is administered in combination with a gastric acid-reducing agent. For example, the subject being administered the gastro-resistant composition is also administered a regimen of a gastric acid-reducing agent. In some embodiments, the gastric-acid reducing agent is administered concomitantly with the gastro-resistant pharmaceutical composition. In some embodiments, the gastric-acid reducing agent is administered sequentially, before or after the gastro-resistant pharmaceutical composition. In some embodiments, the gastric-acid reducing agent is administered at most about 1 hour, or at most about 2 hours, or at most about 3 hours, or at most about 4 hours, or at most about 5 hours, or at most about 6 hours, or at most about 7 hours, or at most about 8 hours, or at most about 9 hours, or at most about 10 hours, or at most about 11 hours, or at most about 12 hours before the gastro-resistant formulation. In some embodiments, the gastric-acid reducing agent is administered at most about 1 hour, or at most about 2 hours, or at most about 3 hours, or at most about 4 hours, or at most about 5 hours, or at most about 6 hours, or at most about 7 hours, or at most about 8 hours, or at most about 9 hours, or at most about 10 hours, or at most about 11 hours, or at most about 12 hours after the gastro-resistant formulation.

In some embodiments, the subject being administered a combination of a gastro-resistant composition and a gastric acid reducing agent is diagnosed with or is exhibiting symptoms of acid reflux disease or gastroesophageal reflux disease (GERD). Non-limiting examples of symptoms of acid reflux disease/GERD include heartburn, indigestion, regurgitation, dyspepsia, bloating, burping, dysphagia, hiccups, nausea, weight loss, wheezing, cough, hoarseness, sore throat, and intestinal bleeding.

In some embodiments, the subject being administered a combination of a gastro-resistant composition and a gastric acid reducing agent is diagnosed with or is exhibiting symptoms of esophagitis. Non-limiting examples of symptoms of esophagitis include difficulties in swallowing, painful swallowing, chest pain, esophageal food impaction, and heartburn and acid reflux.

In some embodiments, the subject being administered a combination of a gastro-resistant composition and a gastric acid reducing agent is diagnosed with or is exhibiting symptoms of peptic ulcer disease. In some embodiments, peptic ulcer disease includes gastric ulcers. In some embodiments, peptic ulcer disease includes duodenal ulcers. Non-limiting examples of symptoms of symptoms of peptic ulcer disease include burning stomach pain, feeling of fullness, bloating or belching, intolerance to fatty foods, heartburn, and nausea.

In some embodiments, the subject being administered a combination of a gastro-resistant composition and a gastric acid reducing agent is diagnosed with or is exhibiting symptoms of Zollinger-Ellison Syndrome. Non-limiting examples of symptoms of symptoms of Zollinger-Ellison Syndrome include nausea, vomiting, weight loss, diarrhea, abdominal pain, heartburn, GERD, and intestinal bleeding.

In some embodiments, the subject being administered a combination of a gastro-resistant composition and a gastric acid reducing agent is diagnosed with or is exhibiting symptoms of Helicobacter pylori infections. Non-limiting examples of symptoms of symptoms of Helicobacter pylori include stomach pain, nausea, loss of appetite, burping, bloating or weight loss.

In some embodiments, the subject being administered a combination of a gastro-resistant composition and a gastric acid reducing agent is administered a gastric acid-reducing agent to reduce a likelihood of occurrence of nonsteroidal anti-inflammatory drug-induced ulcers.

In some embodiments, the pharmaceutical composition is administered in the absence of a gastric acid-reducing agent. For example, the subject being administered the gastro-resistant composition is not administered a regimen of a gastric acid-reducing agent.

In some embodiments, the gastric acid-reducing agent is a proton-pump inhibitor (PPI). Non-limiting examples of proton-pump inhibitors include omeprazole, esomeprazole, lansoprazole, dexlansoprazole, pantoprazole, and rabeprazole.

In some embodiments, the gastric acid-reducing agent is an antacid. Non-limiting examples of antacids include sodium bicarbonate, calcium bicarbonate, aluminum hydroxide, and magnesium hydroxide.

In some embodiments, the gastric acid-reducing agent is a histamine H2 receptor antagonist. Non-limiting embodiments of histamine Ha receptor antagonists include cimetidine, ranitidine, famotidine, and nizatidine.

In some embodiments, the subject administered a gastro-resistant composition is diagnosed with or is exhibiting symptoms of achlorhydria. In some embodiments, the subject administered a gastro-resistant composition is diagnosed with or is exhibiting symptoms of hypochlorhydria. Achlorhydria or hypochlorhydria refer to conditions in which production of hydrochloric acid in the stomach is absent or reduced, respectively. Non-limiting examples of symptoms of symptoms of achlorhydria and hypochlorhydria include epigastric pain, weight loss, heartburn, nausea, bloating, diarrhea, abdominal pain, acid regurgitation, early satiety, vomiting, postprandial fullness, constipation, dysphagia, and glossitis.

In some embodiments, the present disclosure provides a gastro-resistant tablet comprising a tablet core, the tablet core comprising a therapeutically-effective amount of 4-[(7-methoxy-2,3-dihydro-1,4-benzothiazepin-4(5H)yl)methyl]benzoic acid or a pharmaceutically-acceptable salt thereof as an active ingredient, a subcoating layer substantially surrounding the core, and an enteric coating, the enteric coating comprising an enteric polymer, the enteric coating substantially surrounding the subcoat.

In some embodiments, the present disclosure provides a gastro-resistant tablet comprising a tablet core, the tablet core comprising a therapeutically-effective amount of 4-[(7-methoxy-2,3-dihydro-1,4-benzothiazepin-4(5H)yl)methyl]benzoic acid hemifumarate as an active ingredient, a subcoating layer substantially surrounding the core, and an enteric coating, the enteric coating comprising an enteric polymer, the enteric coating substantially surrounding the subcoat.

In some embodiments, the enteric polymer is hydroxypropyl methylcellulose acetate succinate (hypromellose acetate succinate, HPMC-AS), cellulose acetate phthalate, hydroxypropyl methylcellulose phthalate, cellulose acetate trimellitate, polyvinyl acetate phthalate, methacrylic acid/methacrylic acid ester copolymers (e.g., poly(methacrylic acid-co-methyl methacrylate), methacrylic acid/acrylic acid ester copolymers, or shellac (esters of aleurtic acid).

In some embodiments, the enteric coating and the subcoat each provide independently between about 0.1% and about 50% of the mass of the composition, for example, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, or between about 0.1% and about 1%, by mass of the formulation.

The enteric coating and the subcoat can each independently be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% by mass of the formulation.

In some embodiments, the formulation comprises about 1% to about 5% by mass of polymer in the subcoat layer. In some embodiments, the formulation comprises about 1%, about 1.5%, about 2%, about 2.5%, about 3%, about 3.5%, about 4%, about 4.5%, or about 5% or more by mass of a polymer in the subcoat layer.

In some embodiments, the formulation comprises about 5% to about 20% by mass of polymer in the enteric coating layer. In some embodiments, the formulation comprises about 5%, about 6%, about 7%, about 8%, about 9%, about 10%, about 11%, about 12%, about 13%, about 14%, or about 15% or more by mass of a polymer in the enteric coating layer. The pharmaceutical compositions of the present disclosure comprise one or more pharmaceutically-acceptable excipients or carriers. The pharmaceutically-acceptable excipient is provided, for example, as a component of a core, subcoating, or coating layer of the composition. The pharmaceutically-acceptable excipient is, for example, compatible with the other ingredients of the composition and not deleterious to the recipient thereof.

In some embodiments, the pharmaceutical composition is in a form suitable for oral administration. Pharmaceutical compositions for solid oral administration include tablets, dragées, sublingual tablets, sachets, capsules including gelatin capsules, powders, and granules, and those for liquid oral, nasal, buccal, or ocular administration include emulsions, solutions, suspensions, drops, syrups and aerosols. The compounds can also be administered as a suspension or solution via drinking water or with food. In some embodiments, the pharmaceutical composition is in the form of a tablet. In some embodiments, the pharmaceutical composition is in the form of a gastro-resistant tablet.

The pharmaceutically-acceptable excipient or carrier can be selected from various organic and inorganic materials that are used as materials for pharmaceutical formulations. Such materials can be incorporated as any one or more of fillers, diluents, binders, disintegrants, lubricants, glidants, plasticizers, surfactants (wetting agents), buffers (pH adjusting agents), suspending agents, colorants, emulsifiers, flavor-improving agents, gellants, preservatives, solubilizers, stabilizers, sweeteners, tonicity agents, dispersing agents, swelling agents, retardants, absorbents, and/or viscosity-increasing agents.

Non-limiting examples of pharmaceutically-acceptable fillers/diluents include cellulose derivatives including microcrystalline cellulose, silicified microcrystalline cellulose carboxymethyl cellulose, methyl cellulose, hydroxypropyl cellulose, hydroxypropyl methylcellulose, ethyl cellulose, starches, sugars such as mannitol, sucrose, lactose, sorbitol, or dextrins (e.g., maltodextrin), and amino-sugars.

Non-limiting examples of pharmaceutically-acceptable binders include microcrystalline cellulose, gum tragacanth, gelatin, polyvinylpyrrolidone, copovidone, hydroxypropyl methylcellulose, and starch.

Non-limiting examples of pharmaceutically-acceptable disintegrants include croscarmellose sodium, sodium carboxymethyl starch, and crospovidone.

Non-limiting examples of pharmaceutically-acceptable lubricants include stearates such as magnesium stearate and zinc stearate, stearic acid, sodium stearyl fumarate, talc, glyceryl behenate, sodium lauryl sulfate, polyethylene glycol, and hydrogenated vegetable oil.

Non-limiting examples of pharmaceutically-acceptable glidants include colloidal silicon dioxide, talc, tribasic calcium phosphate, calcium silicate, cellulose, magnesium silicate, magnesium trisilicate, starch, magnesium stearate, tale, and mineral oil.

Non-limiting examples of moisture barrier agents include stearic acid.

Non-limiting examples of pharmaceutically-acceptable plasticizers include triethyl citrate.

Non-limiting examples of pharmaceutically-acceptable surfactants include sodium laurylsulfate or polysorbates, polyvinyl alcohol (PVA), polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil, and hardened castor oil such as polyoxyethylene hardened castor oil.

Non-limiting examples of pharmaceutically-acceptable flavoring agents include sweeteners such as sucralose and synthetic flavor oils and flavoring aromatics, natural oils, extracts from plants, leaves, flowers, and fruits, and combinations thereof. Non-limiting examples of flavoring agents include cinnamon oils, oil of wintergreen, peppermint oils, clover oil, hay oil, anise oil, eucalyptus, peppermint, vanilla, citrus oil such as lemon oil, orange oil, grape and grapefruit oil, and fruit essences including apple, peach, pear, strawberry, raspberry, cherry, plum, pineapple, and apricot.

Non-limiting examples of pharmaceutically-acceptable pigments or colorants include alumina (dried aluminum hydroxide), annatto extract, calcium carbonate, canthaxanthin, caramel, B-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, tale, titanium dioxide, aluminum powder, bronze powder, copper powder, and zinc oxide.

Non-limiting examples of buffering or pH adjusting agents include acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide, and magnesium hydroxide.

Non-limiting examples of tonicity enhancing agents include ionic and non-ionic agents such as, alkali metal or alkaline earth metal halides, urea, glycerol, sorbitol, mannitol, propylene glycol, and dextrose.

Non-limiting examples of wetting agents include glycerin, cetyl alcohol, and glycerol monostearate.

Non-limiting examples of preservatives include benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide.

Non-limiting examples of antioxidants include sorbic acid, ascorbic acid, ascorbate, glycine, a-tocopherol, butylated hydroxyanisole (BHA), and butylated hydroxytoluene (BHT).

A pharmaceutically-acceptable excipient can be present in a pharmaceutical composition at a mass of between about 0.1% and about 99% by mass of the composition. For example, a pharmaceutically-acceptable excipient can be present in a pharmaceutical composition at a mass of between about 0.1% and about 95%, between about 0.1% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, between about 0.1% and about 1%, by mass of the formulation.

A pharmaceutically-acceptable excipient can be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about $7%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% by mass of the formulation.

In accordance with the methods of the present disclosure, any of these compounds can be administered to the subject (or are contacted with cells of the subject) in an amount effective to limit or lessen a likelihood of a decrease in the level of RyR-bound Calstabin in the subject, particularly in cells of the subject. Alternatively, the methods of the present disclosure comprise administering a compound in an amount effective to treat or lessen a likelihood of a RyR-related condition as described herein.

The pharmaceutical compositions disclosed herein are suitable for administration to human or animal subjects in a biologically compatible form suitable for administration in vivo. Subjects can be, for example, elderly adults, adults, adolescents, pre-adolescents, children, toddlers, infants, neonates, and non-human animals. In some embodiments, a subject is a patient. In some embodiments, a subject has CPVT. In some embodiments, a subject has CPVT1.

### Dosages and Dosing Regimens

In some embodiments, a suitable amount of 4-[(7-methoxy-2,3-dihydro-1,4-benzothiazepin-4(5H)yl)methyl]benzoic acid or a pharmaceutically-acceptable salt thereof effective to limit or lessen a likelihood of a decrease in the level of RyR-bound Calstabin in the subject and/or to treat or lessen a likelihood of conditions associated with RyR ranges from about 50 to about 500 mg per day, for example about 50 mg per day, about 75 mg per day, about 100 mg per day, about 110 mg per day, about 120 mg per day, about 130 mg per day, about 140 mg per day, about 150 mg per day, about 160 mg per day, about 170 mg per day, about 180 mg per day, about 190 mg per day, about 200 mg per day, about 210 mg per day, about 220 mg per day, about 230 mg per day, about 240 mg per day, about 250 mg per day, about 260 mg per day, about 270 mg per day, about 280 mg per day, about 290 mg per day, about 300 mg per day, about 310 mg per day, about 320 mg per day, about 330 mg per day, about 340 mg per day, about 350 mg per day, about 360 mg per day, about 370 mg per day, about 380 mg per day, about 390 mg per day, about 400 mg per day, about 410 mg per day, about 420 mg per day, about 430 mg per day, about 440 mg per day, about 450 mg per day, about 460 mg per day, about 470 mg per day, about 480 mg per day, about 450 mg per day, or about 500 mg per day.

In some embodiments, a suitable amount of 4-[(7-methoxy-2,3-dihydro-1,4-benzothiazepin-4(5H)yl)methyl]benzoic acid or a pharmaceutically-acceptable salt thereof effective to limit or lessen a likelihood of a decrease in the level of RyR-bound Calstabin in a subject and/or to treat or lessen a likelihood of conditions associated with RyR is about 1 mg to about 2000 mg; from about 1 mg to about 1000 mg; from about 1 mg to about 500 mg; from about 5 mg to about 1000 mg, from about 5 mg to about 500 mg, from about 5 mg to about 100 mg, from about 10 mg to about 50 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

In some embodiments, 4-[(7-methoxy-2,3-dihydro-1,4-benzothiazepin-4(5H)yl)methyl]benzoic acid or a pharmaceutically-acceptable salt thereof is present in a composition in an amount of about 1 mg, about 1.5 mg, about 2 mg, about 2.5 mg, about 3 mg, about 3.5 mg, about 4 mg, about 4.5 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 120 mg, about 125 mg, about 150 mg, about 175 mg, about 200 mg, about 250 mg, about 300 mg, about 350 mg, about 400 mg, about 450 mg, about 500 mg, about $50 mg, about 600 mg, about 650 mg, about 700 mg, about 750 mg, about 800 mg, about 850 mg, about 900 mg, about 950 mg, or about 1000 mg.

In some embodiments, a therapeutically-effective amount of a pharmaceutical composition is administered to a subject, the pharmaceutical composition comprising in a unit dosage form a compound that is 4-[(7-methoxy-2,3-dihydro-1,4-benzothiazepin-4(5H)yl)methyl]benzoic acid or a pharmaceutically-acceptable salt thereof, and a pharmaceutically-acceptable excipient. In some embodiments, the therapeutically-effective amount is about 25 mg per day to about 500 mg per day. In some embodiments, the therapeutically-effective amount is about 50 mg per day to about 500 mg per day, about 100 mg per day to about 500 mg per day, about 150 mg per day to about 500 mg per day, about 200 mg per day to about 500 mg per day, about 250 mg per day to about 500 mg per day, about 300 mg per day to about 500 mg per day, 350 mg per day to about 500 mg per day, about 400 mg per day to about 500 mg per day, about 450 mg per day to about 500 mg per day, about 25 mg per day to about 400 mg per day, about 50 mg per day to about 400 mg per day, about 75 mg per day to about 400 mg per day, about 100 mg per day to about 400 mg per day, about 125 mg per day to about 400 mg per day, about 150 mg per day to about 400 mg per day, about 175 mg per day to about 400 mg per day, about 200 mg per day to about 400 mg per day, about 225 mg per day to about 400 mg per day, about 250 mg per day to about 400 mg per day, about 275 mg per day to about 400 mg per day, about 300 mg per day to about 400 mg per day, about 325 mg per day to about 400 mg per day, about 350 mg per day to about 400 mg per day, about 375 mg per day to about 400 mg per day, about 25 mg per day to about 300 mg per day, about 50 mg per day to about 300 mg per day, about 75 mg per day to about 300 mg per day, about 100 mg per day to about 300 mg per day, about 125 mg per day to about 300 mg per day, about 150 mg per day to about 300 mg per day, about 175 mg per day to about 300 mg per day, about 200 mg per day to about 300 mg per day, about 225 mg per day to about 300 mg per day, about 250 mg per day to about 300 mg per day, about 275 mg per day to about 300 mg per day, about 25 mg per day to about 200 mg per day, about 50 mg per day to about 200 mg per day, about 75 mg per day to about 200 mg per day, about 100 mg per day to about 200 mg per day, about 125 mg per day to about 200 mg per day, about 150 mg per day to about 200 mg per day, or about 175 mg per day to about 200 mg per day. In some embodiments, the therapeutically effective amount is about 25 mg per day, about 50 mg per day, about 75 mg per day, about 100 mg per day, about 125 mg per day, about 150 mg per day, about 175 mg per day, about 200 mg per day, about 225 mg per day, about 250 mg per day, about 275 mg per day, about 300 mg per day, about 325 mg per day, about 350 mg per day, about 375 mg per day, about 400 mg per day, about 425 mg per day, about 450 mg per day, about 475 mg per day, or about 500 mg per day.

In some embodiments, a daily dose of 4-[(7-methoxy-2,3-dihydro-1,4-benzothiazepin-4(5H)yl)methyl]benzoic acid hemifumarate is an amount that is equivalent to 200 mg of 4-[(7-methoxy-2,3-dihydro-1,4-benzothiazepin-4(5H)yl)methyl]benzoic acid. In some embodiments, a daily dose of 4-[(7-methoxy-2,3-dihydro-1,4-benzothiazepin-4(5H)yl)methyl]benzoic acid hemifumarate is an amount that is equivalent to 200 mg of 4-[(7-methoxy-2,3-dihydro-1,4-benzothiazepin-4(5H)yl)methyl]benzoic acid. In some embodiments, a daily dose of 4-[(7-methoxy-2,3-dihydro-1,4-benzothiazepin-4(5H)yl)methyl]benzoic acid hemifumarate is an amount that is equivalent to 300 mg of 4-[(7-methoxy-2,3-dihydro-1,4-benzothiazepin-4(5H)yl)methyl]benzoic acid. In some embodiments, a daily dose of 4-[(7-methoxy-2,3-dihydro-1,4-benzothiazepin-4(5H)yl)methyl]benzoic acid hemifumarate is an amount that is equivalent to 400 mg of 4-[(7-methoxy-2,3-dihydro-1,4-benzothiazepin-4(5H)yl)methyl]benzoic acid. In some embodiments, a daily dose of 4-[(7-methoxy-2,3-dihydro-1,4-benzothiazepin-4(5H)yl)methyl]benzoic acid hemifumarate is 470 mg.

In some embodiments, the present disclosure provides a pharmaceutical composition comprising an amount of 4-[(7-methoxy-2,3-dihydro-1,4-benzothiazepin-4(5H)yl)methyl]benzoic acid hemifumarate equivalent to 20 mg of 4-[(7-methoxy-2,3-dihydro-1,4-benzothiazepin-4(5H)yl)methyl]benzoic acid. In some embodiments, the present disclosure provides a pharmaceutical composition comprising in unit dosage form 23.5 mg of 4-[(7-methoxy-2,3-dihydro-1,4-benzothiazepin-4(5H)yl)methyl]benzoic acid hemifumarate.

In some embodiments, the present disclosure provides a pharmaceutical composition comprising an amount of 4-[(7-methoxy-2,3-dihydro-1,4-benzothiazepin-4(5H)yl)methyl]benzoic acid hemifumarate equivalent to 50 mg of 4-[(7-methoxy-2,3-dihydro-1,4-benzothiazepin-4(5H)yl)methyl]benzoic acid. In some embodiments, the present disclosure provides a pharmaceutical composition comprising in unit dosage form 58.75 mg of 4-[(7-methoxy-2,3-dihydro-1,4-benzothiazepin-4(5H)yl)methyl]benzoic acid hemifumarate.

In some embodiments, the present disclosure provides a pharmaceutical composition comprising an amount of 4-[(7-methoxy-2,3-dihydro-1,4-benzothiazepin-4(5H)yl)methyl]benzoic acid hemifumarate equivalent to 100 mg of 4-[(7-methoxy-2,3-dihydro-1,4-benzothiazepin-4(5H)yl)methyl]benzoic acid. In some embodiments, the present disclosure provides a pharmaceutical composition comprising in unit dosage form 117.5 mg of 4-[(7-methoxy-2,3-dihydro-1,4-benzothiazepin-4(5H)yl)methyl]benzoic acid hemifumarate.

In some embodiments, a therapeutically-effective amount of a pharmaceutical composition is administered to a subject, the pharmaceutical composition comprising in a unit dosage form a compound that is 4-[(7-methoxy-2,3-dihydro-1,4-benzothiazepin-4(5H)yl)methyl]benzoic acid or a pharmaceutically-acceptable salt thereof, and a pharmaceutically-acceptable excipient, wherein the administering is once daily, and the unit dosage form comprises about 20 mg to about 250 mg of 4-[(7-methoxy-2,3-dihydro-1,4-benzothiazepin-4(5H)yl)methyl]benzoic acid hemifumarate. In some embodiments, the unit dosage form comprises about 23.5 mg to about 250 mg, about 25 mg to about 250 mg, about 27.5 mg to about 250 mg, about 30 mg to about 250 mg, about 20 mg to about 235 mg, about 23.5 mg to about 235 mg, about 25 mg to about 235 mg, about 27.5 mg to about 235 mg, or about 30 mg to about 235 mg. In some embodiments, the unit dosage is about 20 mg, about 23.5 mg, about 25 mg, about 27.5 mg, about 30 mg, about 40 mg, about 50 mg, about 52.5 mg, about 55 mg, about 58.75 mg, about 60 mg, about 70 mg, about 80 mg, about 90 mg, about 100 mg, about 110 mg, about 112.5 mg, about 115 mg, about 117.5 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg of 4˜ [(7-methoxy-2,3-dihydro-1,4-benzothiazepin-4(5H)yl)methyl]benzoic acid hemifumarate.

In some embodiments, a therapeutically-effective amount of a pharmaceutical composition is administered to a subject, the pharmaceutical composition comprising in a unit dosage form a compound that is 4-[(7-methoxy-2,3-dihydro-1,4-benzothiazepin-4(5H)yl)methyl]benzoic acid or a pharmaceutically-acceptable salt thereof, and a pharmaceutically-acceptable excipient, wherein the administering is once daily, the unit dosage form comprising an amount of 4-[(7-methoxy-2,3-dihydro-1,4-benzothiazepin-4(5H)yl)methyl]benzoic acid hemifumarate equivalent to about 20 mg to about 250 mg of 4-[(7-methoxy-2,3-dihydro-1,4-benzothiazepin-4(5H)yl)methyl]benzoic acid. In some embodiments, the unit dosage form comprises an amount of 4-[(7-methoxy-2,3˜dihydro-1,4-benzothiazepin-4(5H)yl)methyl]benzoic acid hemifumarate equivalent to about 23.5 mg to about 250 mg, about 25 mg to about 250 mg, about 27.5 mg to about 250 mg, about 30 mg to about 250 mg, about 20 mg to about 200 mg, about 23.5 mg to about 200 mg, about 25 mg to about 200 mg, about 27.5 mg to about 200 mg, or about 30 mg to about 200 mg 4-[(7-methoxy-2,3˜dihydro-1,4-benzothiazepin-4(5H)yl)methyl]benzoic acid In some embodiments, the unit dosage form comprises an amount of 4-[(7-methoxy-2,3-dihydro-1,4-benzothiazepin-4(5H)yl)methyl]benzoic acid hemifumarate equivalent to about 20 mg, about 23.5 mg, about 25 mg, about 27.5 mg, about 30 mg, about 40 mg, about 50 mg, about 52.5 mg, about 55 mg, about 58.75 mg, about 60 mg, about 70 mg, about 80 mg, about 90 mg, about 100 mg, about 110 mg, about 112.5 mg, about 115 mg, about 117.5 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg 4-[(7-methoxy-2,3-dihydro-1,4-benzothiazepin-4(5H)yl)methyl]benzoic acid.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, about 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

In some embodiments, a dose is administered to a subject at a point in time relative to when the subject goes to sleep or wakes from sleep. In some embodiments, a dose is administered at least about 1 hour after the subject wakes. In some embodiments, a dose is administered at least about 2 hours, about 3 hours, about 4 hours, about 5 hours, about 6 hours, about 7 hours, about 8 hours, about 9 hours, about 10 hours, about 11 hours, or about 12 hours after the subject wakes. In some embodiments, a dose is administered from about 1 hour to about 12 hours, about 2 hours to about 12 hours, about 4 hours to about 12 hours, or about 8 hours to about 12 hours after the subject wakes. In some embodiments, a dose is administered at least about 1 hour before the subject goes to sleep. In some embodiments, a dose is administered at least about 2 hours, about 3 hours, about 4 hours, about 5 hours, about 6 hours, about 7 hours, about 8 hours, about 9 hours, about 10 hours, about 11 hours, or about 12 hours before the subject goes to sleep. In some embodiments, a dose is administered from about 1 hour to about 12 hours, about 2 hours to about 12 hours, about 4 hours to about 12 hours, or about 8 hours to about 12 hours before the subject goes to sleep.

### Methods of Preparation

Pharmaceutical compositions described herein can be manufactured by suitable pharmacological techniques. Suitable pharmacological techniques include, e.g., one or a combination of methods such as (1) wet granulation; (2) dry granulation; (3) dry mixing; (4) direct compression; (5) milling; (6) roller compaction; or (7) fusion. Other methods include, e.g., spray drying, pan coating, melt granulation, granulation, fluidized bed spray drying or coating (e.g., wurster coating), tangential coating, top spraying, tableting, and extruding.

In some embodiments, a tablet disclosed herein is prepared by a wet granulation process. In wet granulation, some or all of the active ingredient(s) and excipients in powder form are blended and then further mixed in the presence of a liquid, for example water, that causes the powders to clump into granules. The granulate dried and then screened and/or milled to the desired particle size. The granulate is then tableted, or other excipients can be added prior to tableting, such as a glidant and/or a lubricant.

In some embodiments, an active ingredient, e.g., 4-[(7-methoxy-2,3-dihydro-1,4-benzothiazepin-4(5H)yl)methyl]benzoic acid or a pharmaceutically-acceptable salt thereof is dissolved with one or more pharmaceutically-acceptable excipients, and the resulting mixture is granulated in the presence of a suitable solvent, for example water. A wet granulate is obtained which can be dried and optionally sifted to obtain a dry granulate. The dry granulate can optionally be mixed with one or more additional pharmaceutically-acceptable excipients, optionally sifted, and compressed into tablets.

In some embodiments, a tablet disclosed herein is prepared by a dry granulation process. In some embodiments, a dry granulation process is a slugging process. Slugging is a dry granulation method in which an active ingredient, optionally in combination with one or more excipients, is first compressed to form a slug and is then milled to form particulates suitable for further processing. For example, a blended composition of the active ingredient(s) and pharmaceutically-acceptable excipients may be compacted into a slug or a sheet and then ground into compacted granules. The compacted granules may subsequently be compressed into a tablet. In some embodiments, granulation of an active ingredient, e.g., 4-[(7-methoxy-2,3-dihydro-1,4-benzothiazepin-4(5H)yl)methyl]benzoic acid or a pharmaceutically-acceptable salt thereof may be accomplished by a dry granulation method.

In other embodiments, a blended composition can be compressed directly into a compacted dosage form using direct compression techniques. Direct compression produces a more uniform tablet without granules.

In some embodiments, a granulation method comprises a roller compaction method, in which powder size enlargement is accomplished by feeding an active ingredient, optionally in combination with one or more wet or dry excipients, through a roller apparatus, followed by drying (if necessary), milling, and sizing the compacted mixture to form granules having a desired particle size.

In some embodiments, a capsule described herein can comprise any of the aforementioned blends and granulates described with reference to tableting.

### Numbered Embodiments

Embodiment 1. A method of treating catecholaminergic polymorphic ventricular tachycardia (CPVT), comprising administering to a subject in need thereof a therapeutically-effective amount of a compound that is 4-[(7-methoxy-2,3-dihydro-1,4-benzothiazepin-4(5H)yl)methyl]benzoic acid or a pharmaceutically-acceptable salt thereof.

Embodiment 2. The method of embodiment 1, wherein the administering is once daily.

Embodiment 3. The method of embodiment 1 or embodiment 2, wherein the catecholaminergic polymorphic ventricular tachycardia is catecholaminergic polymorphic ventricular tachycardia type 1.

Embodiment 4. The method of embodiment 3, wherein the catecholaminergic polymorphic ventricular tachycardia type 1 is characterized by a mutation in a Ryanodine Receptor 2 gene.

Embodiment 5. The method of embodiment 4, wherein the mutation in the Ryanodine Receptor 2 gene is an autosomal dominant mutation.

Embodiment 6. The method of any one of embodiments 1-5, wherein the subject is undergoing a treatment regimen for CPVT, wherein the treatment regimen for CPVT comprises a beta-blocker.

Embodiment 7. The method of embodiment 6, wherein the beta-blocker is a non-selective beta-blocker.

Embodiment 8. The method of any one of embodiments 1-7, wherein the subject is undergoing a treatment regimen for CPVT, wherein the treatment regimen for CPVT comprises a sodium channel inhibitor.

Embodiment 9. The method of embodiment 8, wherein the sodium channel inhibitor is flecainide or a pharmaceutically-acceptable salt thereof.

Embodiment 10. The method of any one of embodiments 1-9, wherein the subject is undergoing a treatment regimen for CPVT, wherein the treatment regimen for CPVT comprises use of an implantable cardioverter-defibrillator (ICD).

Embodiment 11. The method of any one of embodiments 1-10, wherein the treating the catecholaminergic polymorphic ventricular tachycardia (CPVT) reduces a likelihood of developing ectopy in the subject.

Embodiment 12. The method of embodiment 11, wherein the ectopy is ventricular ectopy.

Embodiment 13. The method of embodiment 12, wherein the ventricular ectopy is stress-induced.

Embodiment 14. The method of embodiment 12 or embodiment 13, wherein the ventricular ectopy is induced by catecholaminergic stress.

Embodiment 15. The method of any one of embodiments 12-14, wherein the ventricular ectopy is exercise-induced.

Embodiment 16. The method of any one of embodiments 12-15, wherein the ventricular ectopy comprises a premature ventricular contraction.

Embodiment 17. The method of any one of embodiments 12-16, wherein the ventricular ectopy comprises bigeminy.

Embodiment 18. The method of any one of embodiments 12-17, wherein the ventricular ectopy comprises a premature ventricular contraction couplet.

Embodiment 19. The method of any one of embodiments 12-18, wherein the ventricular ectopy comprises tachycardia.

Embodiment 20. The method of embodiment 19, wherein the tachycardia is ventricular tachycardia.

Embodiment 21. The method of any one of embodiments 1-20, wherein the treating the catecholaminergic polymorphic ventricular tachycardia (CPVT) reduces a likelihood of sudden cardiac death in the subject.

Embodiment 22. The method of embodiment 21, wherein the sudden cardiac death is stress-induced.

Embodiment 23. The method of embodiment 21 or embodiment 22, wherein the sudden cardiac death is induced by catecholaminergic stress.

Embodiment 24. The method of any one of embodiments 21-23, wherein the sudden cardiac death is exercise-induced.

Embodiment 25. The method of any one of embodiments 1-24, wherein the treating the catecholaminergic polymorphic ventricular tachycardia (CPVT) reduces a likelihood of atrial fibrillation in the subject.

Embodiment 26. The method of any one of embodiments 1-25, wherein the treating the catecholaminergic polymorphic ventricular tachycardia (CPVT) reduces a likelihood of developing ectopy in the subject while the subject has an elevated heart rate relative to a resting heart rate of the subject.

Embodiment 27. The method of embodiment 26, wherein the elevated heart rate is stress-induced.

Embodiment 28. The method of embodiment 26 or embodiment 27, wherein the elevated heart rate is induced by catecholaminergic stress.

Embodiment 29. The method of any one of embodiments 26-28, wherein the elevated heart rate is exercise-induced.

Embodiment 30. The method of any one of embodiments 1-29, wherein the compound or pharmaceutically-acceptable salt thereof is a hemifumarate salt.

Embodiment 31. The method of any one of embodiments 1-30, wherein the compound is formulated to provide a prolonged release of the compound.

Embodiment 32. The method of embodiment 31, wherein the prolonged release is modified release.

Embodiment 33. The method of embodiment 31, wherein the prolonged release is extended release.

Embodiment 34. The method of embodiment 31, wherein the prolonged release is delayed release.

Embodiment 35. The method of any one of embodiments 1-34, wherein the compound is provided in a solid dosage form.

Embodiment 36. The method of embodiment 35, wherein the solid dosage form is suitable for oral administration.

Embodiment 37. The method of embodiment 35 or embodiment 36, wherein the solid dosage form comprises a pharmaceutically-acceptable excipient.

Embodiment 38. The method of any one of embodiments 1-37, wherein the therapeutically-effective amount is about 50 mg to about 400 mg.

Embodiment 39. The method of any one of embodiments 1-37, wherein the therapeutically-effective amount is about 200 mg.

Embodiment 40. The method of any one of embodiments 1-37, wherein the therapeutically-effective amount is about 300 mg.

Embodiment 41. The method of any one of embodiments 1-37, wherein the therapeutically-effective amount is about 400 mg.

Embodiment 42. The method of any one of embodiments 1-41, wherein the administering is oral.

Embodiment 43. The method of any one of embodiments 1-42, wherein the subject is an adult.

Embodiment 44. The method of any one of embodiments 1-42, wherein the subject is a child.

Embodiment 45. The method of any one of embodiments 1-36, wherein the compound or pharmaceutically acceptable salt thereof is administered to the subject as a pharmaceutical composition in unit dosage form, wherein the unit dosage form further comprises a pharmaceutically acceptable excipient.

Embodiment 46. The method of embodiment 45, wherein in a study, if the unit dosage form is administered to a study subject, then the compound or an ionized form thereof is present in the study subject over a period of time, wherein the period of time occurs after administration, wherein the period of time is at least about 12 hours.

Embodiment 47. The method of embodiment 45, wherein in a study, if the unit dosage form is administered to a study subject, then the compound or an ionized form thereof is present in the study subject over a period of time, wherein the period of time occurs after administration, wherein the period of time is at least about 24 hours.

Embodiment 48. The method of embodiment 46 or embodiment 47, wherein in a study, if the unit dosage form is administered to a study subject, then a maximum plasma concentration of the compound or an ionized form thereof is present in the study subject about 2 to about 6 hours after administration.

Embodiment 49. The method of embodiment 46 or embodiment 47, wherein in a study, if the unit dosage form is administered to a study subject, then a maximum plasma concentration of the compound or an ionized form thereof is present in the study subject about 2 to about 4 hours after administration.

Embodiment 50. The method of embodiment 46 or embodiment 47, wherein in a study, if the unit dosage form is administered to a study subject, then a maximum plasma concentration of the compound or an ionized form thereof is present in the study subject about 3 to about 4 hours after administration.

Embodiment 51. The method of any one of embodiments 45-50, wherein in a study, if the unit dosage form is administered to a study subject, then an in-vivo half-life of the compound or an ionized form thereof of about 14 to about 21 hours is obtained in the study subject.

Embodiment 52. The method of embodiment 51, wherein the in-vivo half-life that is obtained in the study subject is about 14 hours.

Embodiment 53. The method of embodiment 51, wherein the in-vivo half-life that is obtained in the study subject is about 20 hours.

Embodiment 54. The method of any one of embodiments 45-53, wherein in a study, if the unit dosage form is administered to a study subject, then an accumulation ratio for Cₘₐₓ of the compound or an ionized form thereof between about 1.4 and about 1.8 is observed in the study subject, wherein said accumulation ratio is calculated as a ratio of Cₘₐₓ on Day 28/Cₘₐₓ on Day 1, wherein Cₘₐₓ is maximum observed plasma concentration.

Embodiment 55. The method of any one of embodiments 45-54 wherein in a study, if the unit dosage form is administered to a study subject, then an AUC of the compound or an ionized form thereof from about 1.4 to about 1.8 is observed in the study subject, wherein said accumulation ratio for AUC is calculated as a ratio of AUCₜₐᵤ on Day 28/AUC₆₋₂₄ Day 1,
wherein
AUC is area under the concentration-time curve;
AUCₜₐᵤ is area under the concentration-time curve during a dosing interval (tau) at steady-state; and
AUC₀₋₂₄ is area under the concentration-time curve, from time 0 to 24 hours post-dose.

Embodiment 56. The method of any one of embodiments 45-55, wherein in a study, if the unit dosage form is administered to a study subject, then a maximum observed plasma concentration of the compound or an ionized form thereof of no more than about 35 μg/mL is observed in the study subject.

Embodiment 57. The method of any one of embodiments 45-56, wherein in a study, if the unit dosage form is administered to a study subject, then a steady-state plasma concentration of the compound or an ionized form thereof occurs in the study subject in a range of about 3 to about 7 days after administration.

Embodiment 58. The method of any one of embodiments 45-57, wherein the unit dosage form is a tablet.

Embodiment 59. The method of any one of embodiments 45-58, wherein the unit dosage form comprises an amount of 4-[(7-methoxy-2,3-dihydro-1,4-benzothiazepin-4(5H)yl)methyl]benzoic acid hemifumarate equivalent to about 20 to about 200 mg of 4-[(7-methoxy-2,3-dihydro-1,4-benzothiazepin-4(5H)yl)methyl]benzoic acid.

Embodiment 60. The method of any one of embodiments 45-58, wherein the unit dosage form comprises about 23.5 to about 235 mg of 4-[(7-methoxy-2,3-dihydro-1,4-benzothiazepin-4(5H)yl)methyl]benzoic acid hemifumarate.

Embodiment 61. The method of any one of embodiments 45-58, wherein the unit dosage form comprises an amount of 4-[(7-methoxy-2,3-dihydro-1,4-benzothiazepin-4(5H)yl)methyl]benzoic acid hemifumarate equivalent to about 20 mg of 4-[(7-methoxy-2,3-dihydro-1,4-benzothiazepin-4(5H)yl)methyl]benzoic acid.

Embodiment 62. The method of any one of embodiments 45-58, wherein the unit dosage form comprises 23.5 mg of 4-[(7-methoxy-2,3˜dihydro-1,4-benzothiazepin-4(5H)yl)methyl]benzoic acid hemifumarate.

Embodiment 63. The method of any one of embodiments 45-58, wherein the unit dosage form comprises an amount of 4-[(7-methoxy-2,3-dihydro-1,4-benzothiazepin-4(5H)yl)methyl]benzoic acid hemifumarate equivalent to about 50 mg of 4-[(7-methoxy-2,3-dihydro-1,4-benzothiazepin-4(5H)yl)methyl]benzoic acid.

Embodiment 64. The method of any one of embodiments 45-58, wherein the unit dosage form comprises 58.75 mg of 4-[(7-methoxy-2,3-dihydro-1,4-benzothiazepin-4(5H)yl)methyl]benzoic acid hemifumarate.

Embodiment 65. The method of any one of embodiments 45-58, wherein the unit dosage form comprises an amount of 4-[(7-methoxy-2,3-dihydro-1,4-benzothiazepin-4(5H)yl)methyl]benzoic acid hemifumarate equivalent to about 100 mg of 4-[(7-methoxy-2,3-dihydro-1,4-benzothiazepin-4(5H)yl)methyl]benzoic acid.

Embodiment 66. The method of any one of embodiments 45-58, wherein the unit dosage form comprises 117.5 mg of 4-[(7-methoxy-2,3-dihydro-1,4-benzothiazepin-4(5H)yl)methyl]benzoic acid hemifumarate.

Embodiment 67. The method of any one of embodiments 45-66, wherein the unit dosage form is a tablet, wherein the tablet is prepared by a wet granulation process.

Embodiment 68. The method of any one of embodiments 45-66, wherein the unit dosage form is a tablet, wherein the tablet is prepared by a dry granulation process.

Embodiment 69. The method of any one of embodiments 45-68, wherein the unit dosage form is a gastro-resistant tablet.

Embodiment 70. The method of embodiment 69, wherein the gastro-resistant tablet does not substantially disintegrate at a pH not greater than 5.5, wherein disintegration is determined by measuring dissolution of the gastro-resistant tablet in a medium having a pH at or below 5.5.

Embodiment 71. The method of embodiment 70, wherein the medium having a pH at or below 5.5 is 0.1N HCl solution having a pH of 1.2.

Embodiment 72. The method of embodiment 69, wherein the gastro-resistant tablet substantially disintegrates at a pH of about 6.8, wherein disintegration is determined by measuring dissolution of the gastro-resistant tablet in a medium having a pH of 6.8.

Embodiment 73. The method of embodiment 72, wherein the medium having a pH of 6.8 is a phosphate buffer.

Embodiment 74. The method of any one of embodiments 69-73, wherein the gastro-resistant tablet does not substantially disintegrate in gastric fluid.

Embodiment 75. The method of any one of embodiments 69-74, wherein the gastro-resistant tablet substantially disintegrates in intestinal fluid.

Embodiment 76. The method of any one of embodiments 1-75, wherein the subject is in a fed state.

Embodiment 77. The method of any one of embodiments 1-75, wherein the subject is in a fasted state.

Embodiment 78. The method of any one of embodiments 1-77, further comprising administering to the subject a therapeutically-effective amount of a gastric acid-reducing agent.

Embodiment 79. The method of any one of embodiments 1-78, further comprising administering to the subject a therapeutically-effective amount of a gastric acid-reducing agent concurrently with the administering the compound.

Embodiment 80. The method of embodiment 78, wherein the administering the gastric acid-reducing agent is before the administering the compound.

Embodiment 81. The method of embodiment 78, wherein the administering the gastric acid-reducing agent is after the administering the compound.

Embodiment 82. The method of embodiment 78, wherein the administering the gastric acid-reducing agent is performed within about 30 minutes of administering the compound.

Embodiment 83. The method of any one of embodiments 78-82, wherein the gastric acid-reducing agent is a proton-pump inhibitor.

Embodiment 84. The method of any one of embodiments 78-82, wherein the gastric acid-reducing agent is an antacid.

Embodiment 85. The method of any one of embodiments 78-82, wherein the gastric acid-reducing agent is a histamine H2 receptor antagonist.

Embodiment 86. The method of any one of embodiments 1-77, wherein a gastric acid-reducing agent is not administered to the subject within an hour of the administering the compound.

Embodiment 87. The method of any one of embodiments 45-75, wherein the unit dosage form is a gastro-resistant tablet, wherein the gastro-resistant tablet comprises a core and a coating layer substantially covering the core.

Embodiment 88. The method of embodiment 87, wherein the coating layer comprises an enteric polymer.

Embodiment 89. The method of embodiment 88, wherein the enteric polymer is hypromellose acetate succinate.

Embodiment 90. The method of any one of embodiments 87-89, wherein the coating layer is about 20% by mass of the tablet.

Embodiment 91. The method of any one of embodiments 87-90, further comprising a sub-coating layer between the core and the coating layer.

Embodiment 92. The method of embodiment 91, wherein the sub-coating layer comprises a polymer.

Embodiment 93. The method of embodiment 92, wherein the polymer is hypromellose.

Embodiment 94. The method of any one of embodiments 91-93, wherein the sub-coating layer is about 3% by mass of the tablet.

Embodiment 95. The method of any one of embodiments 1-94, further comprising administering to the subject a beta-blocker.

Embodiment 96. The method of embodiment 95, wherein the beta-blocker is administered in an amount that is therapeutically-effective to treat CPVT in the subject in absence of the compound.

Embodiment 97. The method of embodiment 95, wherein the beta-blocker is administered in a reduced amount, wherein the reduced amount is about less than an amount used to treat CPVT in the subject in absence of the compound.

Embodiment 98. The method of any one of embodiments 95-97, wherein the beta-blocker is a non-selective beta-blocker.

Embodiment 99. The method of any one of embodiments 1-98, further comprising administering to the subject a sodium channel inhibitor.

Embodiment 100. The method of embodiment 99, wherein the sodium channel inhibitor is flecainide or a pharmaceutically-acceptable salt thereof.

Embodiment 101. The method of embodiment 99 or embodiment 100, wherein the sodium channel inhibitor is administered in an amount that is therapeutically-effective to treat CPVT in the subject in absence of the compound.

Embodiment 102. The method of embodiment 99 or embodiment 100, wherein the sodium channel inhibitor is administered in a reduced amount, wherein the reduced amount is about less than an amount used to treat CPVT in the subject in absence of the compound.

Embodiment 103. The method of any one of embodiments 1-94, further comprising administering to the subject a beta-blocker and a sodium channel inhibitor.

Embodiment 104. The method of embodiment 103, wherein the sodium channel inhibitor is flecainide or a pharmaceutically-acceptable salt thereof.

Embodiment 105. The method of embodiment 103 or embodiment 104, wherein the beta-blocker is administered in a reduced amount, wherein the reduced amount is about less than an amount used to treat CPVT in the subject in absence of the compound.

Embodiment 106. The method of any one of embodiments 103-105, wherein the sodium channel inhibitor is administered in a reduced amount, wherein the reduced amount is about less than an amount used to treat CPVT in the subject in absence of the compound.

Embodiment 107. The method of any one of embodiments 1-106, wherein the treating increases RyR2-Calstabin2 binding in cardiac muscle of the subject.

Embodiment 108. The method of any one of embodiments 1-107, wherein the treating decreases calcium leak from a RyR2 channel of the subject.

Embodiment 109. The method of any one of embodiments 1-108, wherein the treating decreases open probability (Pa) of RyR2 protein in the subject.

Embodiment 110. A method of treating catecholaminergic polymorphic ventricular tachycardia, comprising administering to a subject in need thereof a therapeutically-effective amount of a pharmaceutical composition, the pharmaceutical composition comprising a tablet, the tablet comprising a core, a sub-coating layer substantially covering the core, and a coating layer substantially covering the sub-coating layer, wherein
the core comprises 4-[(7-methoxy-2,3-dihydro-1,4-benzothiazepin-4(5H)yl)methyl]benzoic acid hemifumarate, mannitol, microcrystalline cellulose, croscarmellose sodium, magnesium stearate, maltodextrin, colloidal anhydrous silica, and sodium stearyl fumarate;
the sub-coating layer comprises hypromellose, microcrystalline cellulose, and stearic acid; and
the coating layer comprises hypromellose acetate succinate, triethyl citrate, sodium lauryl sulfate, and talc,

wherein the administering is once daily.

### EXAMPLES

### Example 1: Gastro-Resistant Tablet

A gastro-resistant tablet comprising 20 mg 4-[(7-methoxy-2,3-dihydro-1,4-benzothiazepin-4(5H)yl)methyl]benzoic acid hemi-fumarate (Compound 1) (based on the mass of 4-[(7-methoxy-2,3-dihydro-1,4-benzothiazepin-4(5H)yl)methyl]benzoic acid) is provided in Table 1.

TABLE 1
Gastro-Resistant Tablet

| Component | | Quantity (mg) | Ratio (%) |
|---|---|---|---|
| Core | Compound 1 | 23.52 | 38.18 |
| | Corresponding in base to | 20 | |
| | Mannitol | 6.78 | 11.01 |
| | Cellulose, microcrystalline | 10.00 | 16.23 |
| | Croscarmellose sodium | 1.50 | 2.44 |
| | Magnesium stearate | 0.50 | 0.81 |
| | Maltodextrin | 7.10 | 11.53 |
| | Silica, colloidal anhydrous | 0.10 | 0.16 |
| | Sodium stearyl fumarate | 0.50 | 0.81 |
| Weight of tablet core | | 81.17 | 50 |
| Sub-coating | Colourless Sepifilm LP 010: | — | 3* |
| | Hypromellose | 1.20 | 1.94 |
| | Cellulose, microcrystalline | 0.15 | 0.24 |
| | Stearic acid | 0.15 | 0.24 |
| | Purified water | q.s.*** | q.s. |
| Enteric | AQOAT suspension: | | 20.2** |
| Coating | Hypromellose acetate | 6.27 | 10.18 |
| | succinate | | |
| | Triethyl citrate | 1.75 | 2.85 |
| | Sodium laurilsulfate | 0.18 | 0.31 |
| | Talc | 1.88 | 3.06 |
| | Purified water | q.s. | q.s. |

| | | |
|---|---|---|
| Total | 61.6 | 100.0 |

*Target mass of coating applied to core tablets. Coating is applied as an 11.2% suspension in purified water containing: hypromellose 80.0%, cellulose microcrystalline 10.0% and stearic acid 10.0%.
**Target mass of coating applied to core tablets. Coating is applied as a 7% AQOAT suspension in purified water containing: Hypromellose acetate succinate (HPMCAS-MF) 7.0%, triethyl citrate 1.96%, sodium laurylsulfate 0.21%, and talc 2.10%.
***q.s. is quantum sufficit (“as much as is sufficient”).

Preparation Method: Compound 1 was mixed with mannitol, cellulose microcrystalline, and maltodextrin. The mixture was granulated via a standard wet granulation process. The obtained wet granulate was dried and sifted. Then, the dry granulate was mixed with croscarmellose, magnesium stearate, silica colloidal anhydrous, and sodium stearyl fumarate. The lubricated granulate was sifted and compressed into tablets.

Subsequently, the core tablets were coated with sub-coating (colourless Sepifilm LP 010). After drying, the enteric coating was applied (AQOAT suspension AS-MF) to obtain enteric-coated tablets.

### Example 2: Placebo

A placebo, enteric-coated tablet formulation is provided in Table 2.

TABLE 2
Placebo

| Ingredients | Amount (g) |
|---|---|
| TABLET CORE | |
| Lactose monohydrate | 30 |
| Microcrystalline cellulose | 19.5 |
| Magnesium stearate | 0.5 |
| For an uncoated tablet with a mass of | 50 |
| SUB-COATING | |
| Sepifilm LP 010, containing*: | |
| Hypromellose 6 cps | 3.9 |
| Hypromellose 15 cps | 1.7 |
| Cellulose microcrystalline | 0.7 |
| Stearic acid | 0.7 |
| ENTERIC COATING | |
| AQOAT suspension, containing**: Hypromellose | 2.7 |
| acetate succinate (HPMCAS-LF) | |
| Triethyl citrate | 0.8 |
| Sodium Lauryl sulphate | 0.1 |
| Talc | 0.8 |
| INTERMEDIARY VEHICLE | |
| Water, purified | q.s |
| For an enteric-coated tablet finished with a mass | 61.6 |

*Coating is applied as an 11.2% sepifilm suspension in purified water containing: hydroxypropylmethyl cellulose (hypromellose) 6 cps 56.0%, hydroxypropylmethyl cellulose 15 cps 24.0%, microcrystalline cellulose 10.0%, stearic acid 10.0%
**Coating is applied as a 7% AQOAT suspension in purified water containing Hypromellose acetate succinate (HPMCAS-LF) 7.0%, Triethyl citrate 2.0%, Sodium Lauryl sulphate 0.21% and Talc 2.10%.

Preparation Method: Lactose monohydrate and cellulose microcrystalline were mixed. Then magnesium stearate was added. Subsequently, the core tablets were coated with sub-coating (colorless Sepifilm LP 010). After drying, the enteric coating was applied (AQUAT suspension AS-MF) to obtain enteric-coated tablets.

### Example 3: Clinical Trial

Catecholaminergic Polymorphic Ventricular Tachycardia (CPVT) is associated with fatal changes in heart rhythms leading to sudden death with stress, exercise or excessive excitement. About two thirds of CPVT1 patients have mutations in the Ryanodine Receptor type 2 (RYR2) gene encoding the RyR2 calcium release channel. The mutations cause leaky RyR2 channels, leading in turn to disease symptoms. This study evaluates the ability of Compound 1 to repair leaky RyR2 and restore normal channel function.

A Phase 2, randomized, double-blinded, placebo-controlled, 2-period crossover clinical trial is performed to determine whether treatment with Compound 1 reduces the likelihood of or decreases exercise-induced ventricular ectopy compared to baseline in subjects with CPVT type 1 (CPVT1). The study also measures safety and tolerability and pharmacokinetics of Compound 1 in subjects with CPVT1. About 20 subjects are randomized into a Compound 1 treatment arm (200 mg daily), and a matching placebo arm, on top of standard of care regimen (e.g., beta blockers and/or sodium channel inhibitor (e.g., flecainide)). Following a washout period of approximately two weeks, subjects switch to the opposite treatment. Each treatment period is about 28-31 days in duration.

### Study Objectives

The primary objective of the study is to determine, using an ectopy complexity scale, whether treatment for about 28 days (up to 31 days) prevents, reduces a likelihood of, or decreases exercise-induced ventricular ectopy compared to baseline in patients with CPVT1.

The secondary objective of this study is to determine safety and tolerability of Compound 1 in patients with CPVT1.

Exploratory objectives of this study are to (i) determine the pharmacokinetics of Compound 1 in subjects with CPVT1; (ii) evaluate an expanded ectopy scale that qualifies both the ectopy and the heart rate at which it occurs, in exercise stress tests (EST); and (iii) to determine the long-term effect on heart rhythm by treatment with Compound 1.

### Study Subjects

Participants have a confirmed genetic diagnosis of CPVT1 and supporting clinical phenotype, including residual ventricular ectopy (a complexity score ≥2; requiring at minimum the presence of PVCs in bigeminy on exercise stress test) on a stable (at least 1 month) standard-of-care, CPVT1-directed treatment regimen which can consist of a beta-blocker, a sodium channel inhibitor (e.g., flecainide), or a combination thereof.

### Study Design

Compound 1 is administered in a gastro-resistant tablet according to Example 1, or placebo according to Example 2, at once-daily doses. The formulation comprises 20 mg Compound 1 (based on the mass of 4-[(7-methoxy-2,3-dihydro-1,4-benzothiazepin-4(5H)yl)methyl]benzoic acid), or matching placebo. Subjects are dosed with 200 mg of Compound 1 (10×20 mg tablets), or matching placebo (10 tablets) daily for approximately 28 days (up to 31 days). Following a washout period of approximately two weeks, subjects switch to the opposite treatment, as described in Table 3.

TABLE 3

| | | |
|---|---|---|
| Arm Title | A | B |
| Arm Type | experimental | placebo |
| [Arm Description] | Subjects receive Compound 1 | Subjects receive Placebo daily |
| | 200 mg/day daily during | during the treatment period |
| | the treatment period | (approximately 28 days). |
| | (approximately 28 days). | Subjects then cross over to |
| | Subjects then cross over to | active for the 2^{nd} period. |
| | placebo for the 2^{nd} period. | |

### Study Endpoints

### Primary Endpoint

The primary endpoint is to assess the effect of Compound 1 on exercise induced ventricular ectopy in participants with CPVT1, using a change in ectopy scoring scale from baseline to approximately day 28 (up to 31 days) versus placebo. The Ectopy Scoring Scale (0-4) is described below:

| | |
|---|---|
| No ectopy | 0 |
| Isolated PVCs | 1 |
| Bigeminy | 2 |
| Couplets | 3 |
| Non-sustained VT | 4 |

### Secondary Endpoint

The Secondary endpoint is a composite safety and tolerability profile. Safety is measured by the frequencies of occurrences of the following: Treatment emergent adverse events (TEAEs)≥grade 2 in severity (CTCAE version 5), all Serious Adverse Events (SAEs), and all Adverse events of special interests (AESIs). Safety and tolerability of Compound 1 are determined by monitoring Adverse Events (AEs) over approximately 28 days of treatment via patient interviews, patient diary reviews, physical examinations, electrocardiograms (ECGs), alerts from continuous cardiac rhythm monitoring during 28-day periods, vital signs, and clinical laboratory safety tests. The Columbia-Suicide Severity Rating Scale (C-SSRS) is administered pre- and post-intervention.

### Exploratory Endpoints

An exploratory endpoint is to evaluate an expanded ectopy scale in exercise stress tests which qualifies both the ectopy and the heart rate at which the ectopy occurs. The Expanded Ectopy Scoring Scale (0-10) is described below:

| | | |
|---|---|---|
| No ectopy | 0 | points |
| PVCs only | 1 | point |
| Bigeminy | 2 | points |
| Couplets | 5 | points |
| Non-sustained VT | 10 | points |

Add 5 points for ectopy onset at heart rate <=120 bpm; 3 points for ectopy onset at heart rate >120 but <=150 bpm; 1 point for ectopy onset at heart rate >150 bpm.

An additional exploratory endpoint is a comparison of a continuous 28-day cardiac monitoring output of Compound 1 treatment versus placebo. Participants are fitted with a continuous cardiac monitoring device at day 1 of each period, prior to first dose. The device is used to monitor cardiac rhythm over the treatment duration (approximately 28 days). The device is then removed at the end of the treatment for each period. The device provides continuous ECG monitoring to monitor for unusual or clinically relevant changes in heart rhythm.

An additional exploratory endpoint is pharmacokinetics (PK) of an approximate 28-days (up to 31 days) administration of compound 1 to study subjects, including (1) last day maximum plasma concentration (Cₘₐₓ), and measurements of the area under the curve (AUC) on the last day of the active treatment period.

The following pharmacokinetic parameters for plasma Compound 1 are calculated on the appropriate blood samples on the last day of treatment (approximately day 28-31):
AUCₜₐᵤ: The area under the concentration-time curve during a dosing interval (tau) at steady state.
Cₘₐₓ: Maximum observed concentration.
Tₘₐₓ: Time to reach Cₘₐₓ. If the maximum value occurs at more than one time point, Tₘₐₓ is defined as the first time point with this value.
Cₘᵢₙ: Minimum observed concentration.
Tₘᵢₙ: Time to reach Cₘᵢₙ.

### Example 4: Preparation of 4-[(7-methoxy-2,3-dihydro-1,4-benzothiazepin-4(5H)yl)methyl]benzoic acid

4-[(7-methoxy-2,3-dihydro-1,4-benzothiazepin-4(5H)yl)methyl]benzoic acid was prepared as described below.

### Stage 1:7-methoxy-2,3,4,5-tetrahydrobenzo[f][1,4]thiazepine (“Amine”)

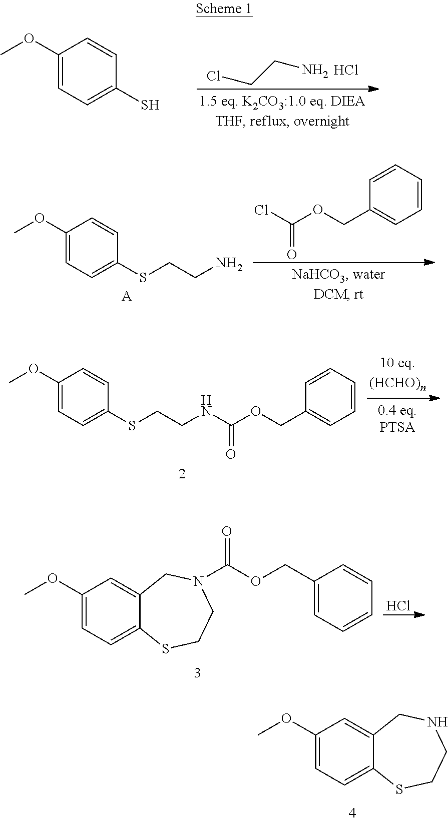

### 2-(4-Methoxyphenylthio) ethanamine (A)

4-Methoxythiophenol (50 g, 0.357 mol), 2-chloroethylamine monohydrochloride (39.8 g, 0.343 mol.), K₂CO₃ (78.8 g, 0.57 mol), and diisopropyl ethylamine (32 mL, 0.178 mol) were mixed in tetrahydrofuran (THF). The mixture was degassed for 5 min. under reduced pressure and heated at reflux under argon overnight. The solvent was removed and water was added to the flask. The mixture was extracted with dichloromethane. The organic layers were collected, dichloromethane was removed and conc. HCl was added, followed by water. The solution was extracted with 1:1 ethyl acetate (EtOAc)/hexane. The aqueous layer was adjusted to pH 10 with 2 M NaOH, and was extracted with dichloromethane. The combined organic solution was dried over anhydrous sodium sulfate. Removal of solvent provided target compound A.

### Benzyl 2-(4-methoxyphenylthio) ethylcarbamate (2)

To a flask containing compound A (8.0 g, 43.7 mmol), sodium bicarbonate (12.1 g, 144 mmol), water, and dichloromethane was added benzyl chloroformate (8.2 g, 48.1 mmol, diluted in 100 mL of dichloromethane) dropwise at 0° C. After the addition, the mixture was stirred at room temperature (r.t.) for 5 hr. The organic layer was collected and the aqueous solution was extracted with 100 mL of dichloromethane. The combined organic solution was dried over sodium sulfate. The solvent was removed and the resulting solid was triturated with THE/hexane (1:10). The solid was collected and dried leaving the target product.

### Benzyl 7-methoxy-2,3-dihydrobenzo[f] [1,4] thiazepine-4(5H)-carboxylate (3)

A mixture of compound 2 (7.3 g, 23 mmol), paraformaldehyde (6.9 g 0.23 mol), and p-toluenesulfonic acid (1.45 g. 7.6 mmol) in toluene was stirred at 70° C. overnight. After cooling to r.t., the solid was filtered off The solution was extracted with saturated sodium carbonate, and the organic layer was dried over anhydrous sodium sulfate to yield the target product as a liquid after removal of the solvent.

### 7-Methoxy-2,3,4,5-tetrahydrobenzo[f][1,4]thiazepine hydrobromide (Amine)

Compound 3 (10 g, 30 mmol) was mixed with conc. HCl, water, and dioxane. The mixture was stirred at 100° C. overnight. After cooling to r.t., most of the solvent and HCl were removed under reduced pressure. Water was added to the solution and the solid was filtered off. The aqueous solution was extracted with EtOAc/hexane (1:1) and basified by adding 15 g of NaOH. The mixture was extracted with dichloromethane. The combined solution was dried over anhydrous sodium sulfate. Removal of solvent provided a liquid that solidified after standing at r.t., to yield the target compound.

### Stage 2: -[(7-methoxy-2,3-dihydro-1,4-benzothiazepin-4(5H)yl)methyl]benzoic acid

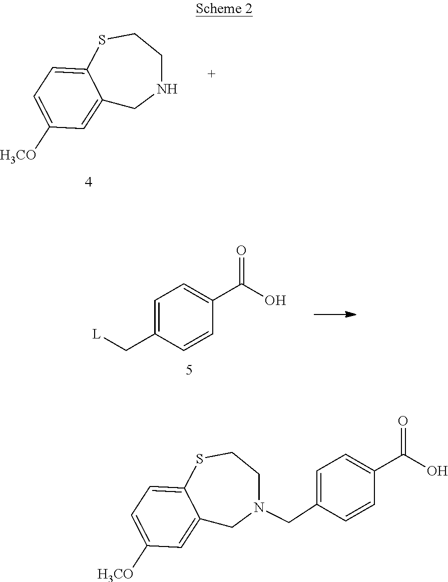

In Scheme 2, L is a leaving group, which is, by way of example, a halogen or a sulfonate (OSO₂R′ wherein R′ is alkyl or aryl, e.g., OMs (mesylate) or OTs (tosylate)). Amine (4) (1 mmol) was dissolved in dichloromethane. To the solution was added alkylation reagent (5) (1 mmol), followed by N,N-diisopropylethylamine (2 mmol). The mixture was stirred at r.t. overnight. The solution was loaded onto a silica gel column directly and eluted with hexane/EtOAc (2:1, v/v) to afford the desired product.

### Preparation of 4-[(7-methoxy-2,3-dihydro-1,4-benzothiazepin-4(5H)yl)methyl]benzoic acid hemifumarate—Form 1

4-[(7-methoxy-2,3-dihydro-1,4-benzothiazepin-4(5H)yl)methyl]benzoic acid was prepared as in Example 1. To form the hemifumarate salt, 4-[(7-methoxy-2,3-dihydro-1,4-benzothiazepin-4(5H)yl)methyl]benzoic acid was salified with fumaric acid in the presence of isopropanol, as depicted in Scheme 3. After cooling, the obtained product was filtered and washed with isopropanol to give the title product.

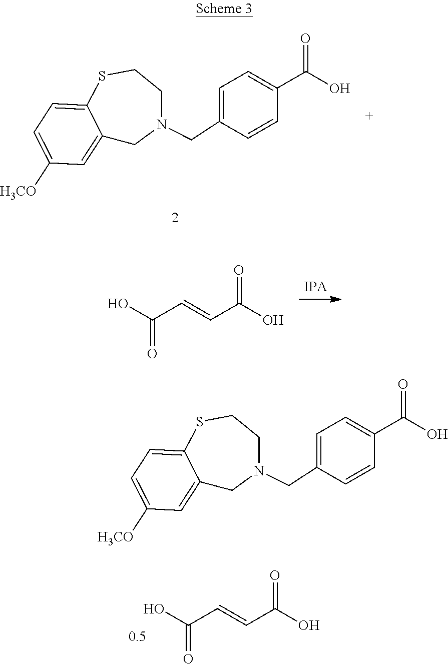 [SMILES: COc1ccc2c(c1)CN(Cc1ccc(C(=O)O)cc1)CCS2 ; O=C(O)/C=C/C(=O)O ; COc1ccc2c(c1)CN(Cc1ccc(C(=O)O)cc1)CCS2 ; O=C(O)/C=C/C(=O)O]

Form 1 can optionally be ground to a particle size distribution described in Table 4.

TABLE 4

| . Particle size | |
|---|---|
| D(v, 0.1) | 3.4 μm ≤ D(v, 0.1) ≤ 4.4 μm |
| −D(v, 0.5) | 6.9 μm ≤ D(v, 0.5) ≤ 9.75 μm |
| −D(v, 0.9) | 13.4 μm ≤ D(v, 0.9) ≤ 19.5 μm |

### Preparation of 4-[(7-methoxy-2,3-dihydro-1,4-benzothiazepin-4(5H)yl)methyl]benzoic acid hemifumarate-Form 2

4-[(7-methoxy-2,3-dihydro-1,4-benzothiazepin-4(5H)yl)methyl]benzoic acid was prepared as in Example 1. To form the hemifumarate salt, 4˜ [(7-methoxy-2,3-dihydro-1,4-benzothiazepin-4(5H)yl)methyl]benzoic acid was salified with fumaric acid in the presence of a mixture of dimethylsulfoxide and water, as depicted in Scheme 4. After cooling, the obtained product was filtered and washed with water and acetone to give the desired product.

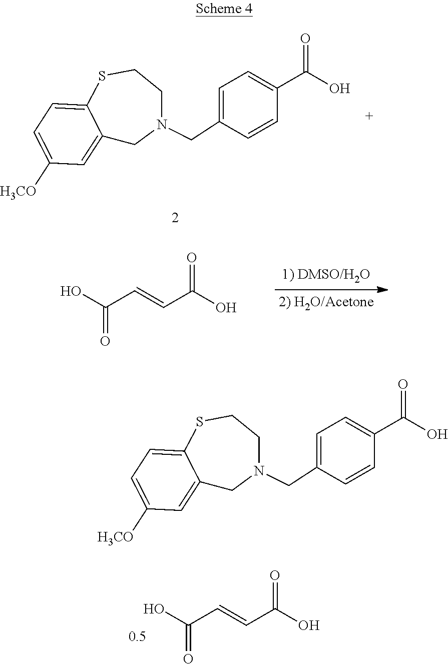 [SMILES: COc1ccc2c(c1)CN(Cc1ccc(C(=O)O)cc1)CCS2 ; O=C(O)/C=C/C(=O)O ; COc1ccc2c(c1)CN(Cc1ccc(C(=O)O)cc1)CCS2 ; O=C(O)/C=C/C(=O)O]

Form 2 can optionally be ground to a particle size distribution described in Table 5.

TABLE 5

| . Particle size | |
|---|---|
| −D(v, 0.5) | 10 μm ≤ D(v, 0.5) ≤ 30 μm |
| −D(v, 0.9) | ≤90 μm |

### Example 5: Gastro-Resistant Tablet (Formulation A)

A gastro-resistant tablet comprising 20, 40, or 200 mg 4-[(7-methoxy-2,3-dihydro-1,4-benzothiazepin-4(5H)yl)methyl]benzoic acid hemifumarate (Compound 1) (based on the mass of 4-[(7-methoxy-2,3-dihydro-1,4-benzothiazepin-4(5H)yl)methyl]benzoic acid) is provided in Table 6.

TABLE 6
Gastro-Resistant Tablet

| | | Quantity/Unit (mg/tablet) | | |
|---|---|---|---|---|
| Component | | 20 mg | 40 mg | 200 mg |
| Intra-granular | Compound 1 | 23.52 | 47.04 | 235.20 |
| | Corresponding in base to | 20 | 40 | 200 |
| | Cellulose, microcrystalline | 8.48 | 16.96 | 84.82 |
| | Povidone (PVP K17) | 5.20 | 10.40 | 52.00 |
| | Crospovidone | 1.00 | 2.00 | 10.00 |
| | Polysorbate 80 | 0.12 | 0.24 | 1.18 |
| | Purified water | q.s. | q.s. | q.s. |
| Extra-granular | Crospovidone | 1.20 | 2.40 | 12.00 |
| | Magnesium stearate | 0.40 | 0.80 | 4.00 |
| | Silica, colloidal anhydrous | 0.08 | 0.16 | 0.80 |
| Weight of tablet core (mg) | | 40 | 80 | 400 |
| Sub-coating | White Sepifilm LP 770, containing: | Target 3%* | Target 3%* | Target 3%* |
| | Hypromellose | 0.78 | 1.56 | 7.80 |
| | Cellulose, microcrystalline | 0.12 | 0.24 | 1.20 |
| | Stearic acid | 0.12 | 0.24 | 1.20 |
| | Titanium dioxide E171 | 0.18 | 0.36 | 1.80 |
| | Purified water | q.s.*** | q.s. | q.s. |
| Enteric | AQOAT suspension, containing: | Target 21%** | Target 17%** | Target 10%** |
| Coating | Hypromellose acetate succinate | 5.49 | 8.89 | 26.14 |
| | (HPMCAS-LF) | | | |
| | Triethyl citrate | 1.10 | 1.78 | 5.23 |
| | Sodium laurilsulfate | 0.16 | 0.27 | 0.78 |
| | Talc | 1.65 | 2.67 | 7.84 |
| | Purified water | q.s. | q.s. | q.s. |

| | | | |
|---|---|---|---|
| Total Weight of tablets (mg) | 49.6 | Approx. 96 | Approx. 452 |

*Target mass of coating applied to core tablets. Coating is applied as an 11.2% suspension in purified water containing: hypromellose 65.0%, cellulose microcrystalline 10.0%, stearic acid 10.0%, and titanium dioxide E171 15.0%.
**Target mass of coating applied to core tablets. Coating is applied as a 7% AQOAT suspension in purified water containing: hypromellose acetate succinate (HPMCAS-LF) 7.0%, triethyl citrate 1.40%, sodium laurilsulfate 0.21%, and talc 2.10%.
***q.s. is quantum sufficit (“as much as is sufficient”).

Preparation Method: Compound 1 was mixed with cellulose microcrystalline, povidone and crospovidone. The mixture was granulated using a standard wet granulation process. Polysorbate was added to purified water to function as a granulation fluid. The wet granulate was dried in an oven system and sifted. The dry granulate was mixed with the external phase: crospovidone, magnesium stearate, and silica colloidal anhydrous, and the lubricated granulate was sifted and compressed into tablets.

Subsequently, the core tablets were coated with a premix (white Sepifilm LP 770) (sub-coating). After drying, the enteric coating is applied as an AQOAT suspension to obtain gastro-resistant tablets.

### Example 6: Clinical Trial in Healthy Volunteers

A Phase 1, randomized, double-blind, placebo-controlled clinical trial was performed to assess the safety and pharmacokinetics (PK) of Compound 1 after single, escalating, and repeated oral doses in healthy male volunteers.

### Study Objectives

### Part I (Single Ascending Doses—SAD)

The primary objective was to assess the safety of escalating single oral doses of Compound 1 compared with placebo in healthy male volunteers. The secondary objective was to measure plasma pharmacokinetic (PK) parameters of Compound 1.

### Part II (Multiple Ascending Doses—MAD)

The primary objective was to assess the safety of escalating repeated oral doses of Compound 1 over 14 days compared with placebo in healthy male volunteers. The secondary objective was to measure plasma pharmacokinetic (PK) parameters of Compound 1 and to assess the concentration of Compound 1 in quadriceps muscle.

### Materials and Methods

### 1. Study Drug

4-[(7-methoxy-2,3-dihydro-1,4-benzothiazepin-4(5H)yl)methyl]benzoic acid hemi-fumarate (Compound 1) was administered in a gastro-resistant tablet according to EXAMPLE 5. The formulation contained 20 mg, 40 mg, or 200 mg Compound 1 (based on the mass of 4-[(7-methoxy-2,3-dihydro-1,4-benzothiazepin-4(5H)yl)methyl]benzoic acid). Placebo tablet according to Example 2 was used as comparator.

### 2. Study Design

The study was subdivided into two parts (I and II). Each part was randomized, double-blind, placebo-controlled.

In part I, participants from groups A, B, C, D, and E received single doses of 40 mg, 80 mg, 160 mg, 240 mg, or 400 mg Compound 1, respectively, or corresponding placebo. Forty subjects completed part I: 30 active (6 per cohort) and 10 placebo.

In Part II, participants from groups I, J, K, and L received repeated doses of 20 mg, 60 mg, 120 mg, or 240 mg Compound 1, respectively, or corresponding placebo once daily over a period of 14 days. Forty-one subjects completed Part II: 7/7/8/7 for cohorts I, J, K, L, and twelve placebo.

### 3. Pharmacokinetic Measurements

3a. Blood Sampling:

In Part I, blood samples were collected from each participant as follows: at pre-dose, then 0.25, 0.5, 1, 1.5, 2, 3, 4, 5, 6, 8, 10, 12, 16, 24, 36, 48, 60, 72, and 96 hours after dosing.

In Part II, blood samples were collected from each participant as follows: Day 1:12 samples from the pre-morning dose up to 16 h (pre-dose, 0.25, 0.5, 1, 1.5, 2, 3, 4, 6, 8, 12, and 16 h), Days 2-13:12 samples at pre-doses (i.e., morning dose); Day 14:16 samples from the pre-morning dose up to 96 (D14 pre-dose, 0.25, 0.5, 1, 1.5, 2, 3, 4, 6, 8, 12, and 16 h and D15-18:1 sample before theoretical morning dosing.

The following pharmacokinetic parameters were calculated from plasma concentration time profiles.
AUCₗₐₛₜ (AUClast): area under the concentration-time curve from time zero (time of drug administration) to tₗₐₛₜ
AUC: area under the concentration-time curve from time zero (time of drug administration) to infinity
AUC₂₄ (AUC24): area under the concentration-time curve from time zero to 24h on Day 1.
AUC_{τ} (AUCₜₐᵤ): area under the concentration-time curve over the dosing interval at steady state on Day 14.
Cₘₐₓ (Cₘₐₓ): maximum observed concentration
C_{maxds} (Cmaxds): Cₘₐₓ/dose
tₘₐₓ (tₘₐₓ): time corresponding to Cₘₐₓ
t_{tag} (tlag): lag time: time prior to the time corresponding to the first measurable concentration
Cₗₐₛₜ (Clast): last quantifiable observed concentration
R_{ac(AUCτ)} (RacAUC): accumulation ratio of AUC (AUC_{τ}/AUC₂₄)
R_{acCmax} (RacCmax): accumulation ratio of Cₘₐₓ (Cₘₐₓ Day 14/Cₘₐₓ Day 1)
tₗₐₛₜ (tlast): time corresponding to Clast
λ_{z} (k): first order rate constant of the terminal phase
t_{1/2,z} (t½): terminal elimination half-life of the compound, calculated as: t_{1/2,z}=ln(2)/λ_{z} (λ_{z} being the rate constant of the terminal phase).

The area under the plasma concentration-time curve was calculated using a combined linear and logarithmic trapezoidal rule. The interpolation was linear in the constant and ascending parts of the plasma concentration-time profiles. The interpolation was logarithmic in the descending parts.

For Part I, to assess the dose effect on the PK of Compound 1, the mean AUCₗₐₛₜ, and Cₘₐₓ for each dose were calculated and were plotted by dose.

For Part II, the same approach was used on AUC, (Day 14) respectively AUC₂₄ (Day 1), and Cₘₐₓ.

3b. Muscle Biopsy

For Study part II (for all groups), Concentrations of Compound 1 were determined in muscle by performing muscle biopsy on quadriceps muscle on day 13, at least 3 hours after morning dose.

3c. Statistical Methods:

For each group and treatment, descriptive statistics (N, arithmetic mean, standard deviation, minimum, median, maximum, coefficient of variation, geometric mean, and geometric CV, as appropriate) were calculated for the concentrations and pharmacokinetic parameters of Compound 1.

3d. Analytical methods:

Plasma and muscle samples were analyzed by LC-MS/MS method.

### Results

### 1. Pharmacokinetics

1a. Part I: Single Ascending Dose

Mean concentration time profiles of Part I (single dose) are shown in FIG. 1. Concentrations are shown in ng/mL.

Table 7 summarizes pharmacokinetic parameters after a single dose (arithmetic and geometric means, standard deviation (SD), coefficient of variation (CV %) and geometric CV %, median and range).

TABLE 7
Part I: AUCₗₐₛₜ, AUC, Cₘₐₓ, and tₘₐₓ on Day 1

| | Treatment A | | Treatment B | | Treatment C | | Treatment D | | Treatment E | |
|---|---|---|---|---|---|---|---|---|---|---|
| | (n = 6)/40 mg | | (n = 6)/80 mg | | (n = 6)/160 mg | | (n = 6)/240 mg | | (n = 6)/400 mg | |
| Part I | Geom. Mean | | Geom. Mean | | Geom. Mean | | Geom. Mean | | Geom. Mean | |
| Parameter/Unit | (geom. % CV) | | (geom. % CV) | | (geom. % CV) | | (geom. % CV) | | (geom. % CV) | |
| AUCₗₐₛₜ | 68658 | (16.3) | 152782 | (6.8) | 303849 | (19.7) | 450902 | (20.9) | 656199 | (12.0) |
| ng*h/mL | | | | | | | | | | |
| AUC | 71710 | (17.8) | 162902 | (7.3) | 312443 | (20.5) | 463877 | (23.0) | 708394 | (14.0) |
| ng*h/mL | | | | | | | | | | |
| Cₘₐₓ | 3940 | (3.4) | 6883 | (32.4) | 15490 | (13.1) | 24461 | (8.5) | 35101 | (5.8) |
| ng/mL | | | | | | | | | | |
| t_{1/2, z} | 20.9 | (12.3) | 22.7 | (15.4) | 17.5 | (14.6) | 17.7 | (16.8) | 18.7 | (12.6) |
| h | | | | | | | | | | |
| tₘₐₓ# | 3.00 | (2.0-5.0) | 3.00 | (1.5-12.0) | 3.00 | (2.0-5.0) | 4.50 | (2.0-5.0) | 3.50 | (3.0-5.0) |
| h | | | | | | | | | | |

#median and range

1b. Part II: Multiple Ascending Dose

Mean concentration time profiles of Part II (repeated doses) on day 1 are shown in FIG. 2. Concentrations are shown in ng/mL.

Mean concentration time profiles of Part II (repeated doses) on day 14 are shown in FIG. 3. Concentrations are shown in ng/mL.X

Table 8 summarizes pharmacokinetic parameters after repeated doses (arithmetic and geometric means, SD, CV % and geometric CV %, median and range).

TABLE 8
Part II: AUC₀₋₂₄, AUC_{τ}, Cₘₐₓ, and tₘₐₓ on Day 1 and on Day 14

| | | Treatment I | Treatment J | Treatment K | Treatment L |
|---|---|---|---|---|---|
| | | (n = 7)/20 mg | (n = 7)/60 mg | (n = 8)/120 mg | (n = 7)/240 mg |
| Part II, Day 1 and Day 14 | | Geom. Mean | Geom. Mean | Geom. Mean | Geom. Mean |
| Parameter | Unit | (geom. % CV) | (geom. % CV) | (geom. % CV) | (geom. % CV) |

Day 1

| | | | | | | | | | |
|---|---|---|---|---|---|---|---|---|---|
| AUC₀₋₂₄ | ng*h/mL | 24342 | (19.3)* | 69212 | (16.5) | 138083 | (10.5) | 270249 | (13.0) |

| | | | | | | | | |
|---|---|---|---|---|---|---|---|---|
| Cₘₐₓ | ng/mL | 2201 (20.4) | 6091 | (14.4) | 11478 | (11.1) | 23863 | (7.9) |
| | | (n = 8) | | | | | | |
| tₘₐₓ# | h | 2.52 (2.0-6.0) | 3.00 | (1.5-4.0) | 3.00 | (2.0-6.0) | 3.00 | (3.0-4.0) |
| | | (n = 8) | | | | | | |

Day 14

| | | | | | | | | | |
|---|---|---|---|---|---|---|---|---|---|
| AUC_{τ} | ng*h/mL | 40124 | (21.3 | 115218 | (19.7) | 220525 | (22.3) | 464565 | (14.0) |
| Cₘₐₓ | ng/mL | 3107 | (13.6) | 8835 | (12.7) | 16341 | (17.6) | 34852 | (14.7) |
| t_{1/2, z} | h | 19.3 | (22.2) | 19.7 | (15.4) | 19.3 | (19.8) | 19.8 | (11.3) |
| Rac_{(AUCτ)} | — | 1.65 | (15.4) | 1.66 | (12.5) | 1.60 | (12.8) | 1.72 | (10.5) |
| Rac_{(Cmax)} | — | 1.39 | (17.3) | 1.66 | (12.5) | 1.42 | (8.8) | 1.46 | (13.6) |
| tₘₐₓ# | h | 3.00 | (1.5-6.0) | 1.45 | (15.1) | 3.50 | (1.5-6.0) | 3.00 | (2.0-4.0) |

#median and range
*n = 7, one withdrawn subject

### 2. Muscle Biopsy

Muscle biopsy was performed to assess the concentration of Compound 1 in quadriceps muscle. The biopsy was performed on Day 13 at 3 hours post dose at least.

Table 9 shows the concentrations of Compound 1 in the muscle summarized by medians and ranges.

TABLE 9
Concentrations of Compound 1 in quadriceps muscle (unbound and total)

| | | Treatment I | Treatment J | Treatment K | Treatment L |
|---|---|---|---|---|---|
| | | (n = 7)/20 mg | (n = 7)/60 mg | (n = 8)/120 mg | (n = 7)/240 mg |
| Part II | | Median | Median | Median | Median |
| Parameter | Unit | (Range) | (Range) | (Range) | (Range) |
| Unbound Cpd I | ng/g | 22.0 (17.5-41.5) | 80.3 (49.7-124) | 104 (63.2-196) | 242 (196-452) |
| concentration | | | | | |
| Total Cpd I | ng/g | 171 (141-341) | 489 (273-693) | 913 (614-1207) | 1480 (890-1745) |
| concentration | | | | | |

Table 10 summarizes the ratios of unbound respective total concentration at Day 13 at 3 hours post dose divided by the plasma concentration on Day 14 at 3 hours post dose.

TABLE 10
Ratios of Compound 1 concentrations in quadriceps muscle
(unbound and total) divided by plasma concentrations

| | | Treatment I | Treatment J | Treatment K | Treatment L |
|---|---|---|---|---|---|
| | | (n = 7)/20 mg | (n = 7)/60 mg | (n = 8)/120 mg | (n = 7)/240 mg |
| Part II | | Geom. Mean | Geom. Mean | Geom. Mean | Geom. Mean |
| Parameter | Unit | (geom. % CV) | (geom. % CV) | (geom. % CV) | (geom. % CV) |
| Kp | (ng/g)/(ng/mL) | 0.0808 (65.3) | 0.0640 (22.6) | 0.0636 (49.9) | 0.0440 (30.0) |
| Kpu | (ng/g)/(ng/mL) | 0.0106 (59.2) | 0.0100 (31.3) | 0.0081 (60.7) | 0.0081 (32.5) |

### 3. Discussion:

Part I: After single oral administration in young healthy males of 20, 40, 80, 160, 240, and 400 mg of 4-[(7-methoxy-2,3-dihydro-1,4-benzothiazepin-4(5H)yl)methyl]benzoic acid hemifumarate, rapid absorption was observed, after a lag time consistent with the gastro-resistant (GR) properties of the tablet administered. No evidence of non-linearity with the dose was observed. The inter-individual variability across treatment groups was low. Geometric mean tin ranged from 17.5 h in the 160 mg cohort to 22.7 h in the 80 mg cohort while the geometric % CV ranged from 12.3% to 16.8%. Graphical inspections show dose proportionality with regard to AUCₗₐₛₜ and Cₘₐₓ.

Part II: After 14-days of repeated once daily oral administrations of 20 mg of 4-[(7-methoxy-2,3-dihydro-1,4-benzothiazepin-4(5H)yl)methyl]benzoic acid hemifumarate, Cₘₐₓ was reached with a median Tₘₐₓ between 3 and 3.5 hours. Steady-state was attained after 3-7 days of daily treatment (e.g., at day 4 in the 20, 60, and 120 mg cohorts and at day 5 in the 240 mg cohort). The geometric % CV for the AUC on Days 1 and 14 ranged from 10.5% (120 mg cohort, Day 1) to 22.3% (120 mg cohort, Day 14). The inter-individual variability across treatment groups was low. Geometric mean fuzz at Day 14 ranged from 19.3 h in the 20 and 120 mg cohorts to 19.8 h in the 240 mg cohort. The % CV ranged from 11.3% to 22.2%. Graphical inspections showed dose proportionality regarding AUC, resp. AUC₀₋₂₄ and Cₘₐₓ. Geometric mean accumulation ratios based on AUC ranging from 1.60 to 1.72 and on Cₘₐₓ ranging from 1.39 to 1.46 appeared similar across dose groups.

The median unbound and total 4-[(7-methoxy-2,3-dihydro-1,4-benzothiazepin-4(5H)yl)methyl]benzoic acid hemifumarate concentrations in quadriceps muscle appeared dose proportional. The geometric mean ratios of concentrations in muscle divided by plasma concentrations ranged from 0.0440 to 0.0808 (ng/g)/(ng/ml.) for Kp and from 0,0081 to 0.0106 (ng/g)/(ng/mL) for Kpu. The geometric % CVs ranged from 22.6% to 65.3% for Kp and from 31.3% to 60.7% for Kpu.

### 4. Safety

No serious adverse events were reported. The compound was well tolerated at the doses and dose regimens tested.

### 5. Summary and Conclusions

After single oral administration of 4-[(7-methoxy-2,3-dihydro-1,4-benzothiazepin-4(5H)yl)methyl]benzoic acid hemifumarate at 20 to 400 mg, Coax was reached with median Tₘₐₓ values between 3 and 4.5 hrs, after a t_{lag} consistent with the gastro-resistant properties of the tablet (less than 2 hours).

After 14-days of repeated oral administrations of 4-[(7-methoxy-2,3-dihydro-1,4-benzothiazepin-4(5H)yl)methyl]benzoic acid hemifumarate at 20, 60, 120, and 240 mg/d, the Cₘₐₓ was reached with a median Tₘₐₓ between 3 and 4 hours. Mean apparent terminal half-life was around 20 hrs based on the results of the single and repeat dose studies. No evidence of non-linearity with the time and dose was observed. Overall, inter-individual variability on Cₘₐₓ and AUC24 was low at 20, 60, 120, and 240 mg/d. Steady-state was attained after 5 to 7 days of daily treatment. After 14-day repeated administrations of 20, 60, 120, and 240 mg/d of 4-[(7-methoxy-2,3-dihydro-1,4-benzothiazepin-4(5H)yl)methyl]benzoic acid hemifumarate, accumulation ratios were around 1.4-1.8 for Cₘₐₓ and AUC₂₄, consistent with the half-life of Compound 1 and the dose regimen. Kₚ and Kₚᵤ results are concordant within the dose range (20-240 mg) with [0.04-0.08] and [0.008-0.01] values, respectively.

4-[(7-methoxy-2,3-dihydro-1,4-benzothiazepin-4(5H)yl)methyl]benzoic acid hemifumarate was well tolerated at the doses and dose regimens tested.

1. A method of treating catecholaminergic polymorphic ventricular tachycardia (CPVT), comprising administering to a subject in need thereof a therapeutically-effective amount of a compound that is 4-[(7-methoxy-2,3-dihydro-1,4-benzothiazepin-4(5H)yl)methyl]benzoic acid or a pharmaceutically-acceptable salt thereof, wherein the administering is once daily.

2. The method of claim 1, wherein the catecholaminergic polymorphic ventricular tachycardia is catecholaminergic polymorphic ventricular tachycardia type 1.

3. The method of claim 2, wherein the catecholaminergic polymorphic ventricular tachycardia type 1 is characterized by a mutation in a Ryanodine Receptor 2 gene.

4. The method of claim 3, wherein the mutation in the Ryanodine Receptor 2 gene is an autosomal dominant mutation.

5. The method of claim 1, wherein the subject is undergoing a treatment regimen for CPVT, wherein the treatment regimen for CPVT comprises a beta-blocker.

6. (canceled)

7. The method of claim 1, wherein the subject is undergoing a treatment regimen for CPVT, wherein the treatment regimen for CPVT comprises a sodium channel inhibitor.

8. The method of claim 7, wherein the sodium channel inhibitor is flecainide or a pharmaceutically-acceptable salt thereof.

9. (canceled)

10. The method of claim 1, wherein the treating the catecholaminergic polymorphic ventricular tachycardia (CPVT) reduces a likelihood of developing ectopy in the subject.

11-19. (canceled)

20. The method of claim 1, wherein the treating the catecholaminergic polymorphic ventricular tachycardia (CPVT) reduces a likelihood of sudden cardiac death in the subject.

21-28. (canceled)

29. The method of claim 1, wherein the compound or pharmaceutically-acceptable salt thereof is a hemifumarate salt.

30-43. (canceled)

44. The method of claim 1, wherein the compound or pharmaceutically acceptable salt thereof is administered to the subject as a pharmaceutical composition in unit dosage form, wherein the unit dosage form further comprises a pharmaceutically acceptable excipient.

45-47. (canceled)

48. The method of claim 44, wherein the unit dosage form comprises an amount of 4-[(7-methoxy-2,3-dihydro-1,4-benzothiazepin-4(5H)yl)methyl]benzoic acid hemifumarate equivalent to about 20 to about 200 mg of 4-[(7-methoxy-2,3-dihydro-1,4-benzothiazepin-4(5H)yl)methyl]benzoic acid.

49-57. (canceled)

58. The method of claim 44, wherein the unit dosage form is a gastro-resistant tablet.

59-76. (canceled)

77. The method of claim 1, further comprising administering to the subject a beta-blocker.

78. (canceled)

79. The method of claim 77, wherein the beta-blocker is administered in a reduced amount, wherein the reduced amount is about less than an amount used to treat CPVT in the subject in absence of the compound.

80. The method of claim 77, wherein the beta-blocker is a non-selective beta-blocker.

81. The method of claim 1, further comprising administering to the subject a sodium channel inhibitor.

82. The method of claim 81, wherein the sodium channel inhibitor is flecainide or a pharmaceutically-acceptable salt thereof.

83. (canceled)

84. The method of claim 81, wherein the sodium channel inhibitor is administered in a reduced amount, wherein the reduced amount is about less than an amount used to treat CPVT in the subject in the absence of the compound.

85-91. (canceled)

92. A pharmaceutical composition comprising in a unit dosage form 4-[(7-methoxy-2,3-dihydro-1,4-benzothiazepin-4(5H)yl)methyl]benzoic acid or a pharmaceutically-acceptable salt thereof, wherein in a controlled study, if the unit dosage form is administered to a study subject, then an accumulation ratio for AUC of 4-[(7-methoxy-2,3-dihydro-1,4-benzothiazepin-4(5H)yl)methyl]benzoic acid or the pharmaceutically-acceptable salt thereof or a pharmaceutically-acceptable ion thereof between about 1.4 and about 1.8 is present in the subject, wherein the controlled study comprises:
(a) orally administering the pharmaceutical composition in unit dosage form to the study subject;
(b) after the administering, collecting blood samples from the study subject at one or more time points after the administering; and
(c) measuring a plasma concentration of 4-[(7-methoxy-2,3-dihydro-1,4-benzothiazepin-4(5H)yl)methyl]benzoic acid or an ionized form thereof in the blood samples,
wherein the accumulation ratio for AUC is calculated as a ratio of AUC₀₋₂₄ at steady state/AUC₀₋₂₄ Day 1,
wherein:
AUC is area under the concentration-time curve; and
AUC₀₋₂₄ is area under the concentration-time curve, from time 0 to 24 hours post-dose.
